(19) United States
(12) Patent Application Publication
(10) Pub. No.: US 2007/0197609 A1
(43) Pub. Date: Aug. 23, 2007

(54) SALTS OF INDUCIBLE NITRIC OXIDE SYNTHASE DIMERIZATION INHIBITORS

(75) Inventors: Brett A. Cowans, San Diege, CA (US); Patricia Mougin-Andres, San Diego, CA (US); Patrick E. Wheeler, San Diego, CA (US); Timothy C. Gahman, Encinitas, CA (US); Mark R. Herbert, San Diego, CA (US)

Correspondence Address: GLOBAL PATENT GROUP;ATTN: MS LAVERN HALL, P.O. BOX 38100, ST. LOUIS, MO, 63138, US

(73) Assignee: KALYPSYS, INC., San Diego, CA (US)

(21) Appl. No.: 11/563,194

(22) Filed: Nov. 25, 2006

Related U.S. Application Data
(60) Provisional application No. 60/740,322, filed on Nov. 28, 2005.

(51) Int. Cl. A61K 31/433; C07D 417/14

(52) U.S. Cl. 514/362; 548/128

(57) ABSTRACT

The present invention relates to novel salts and methods useful as inhibitors of the nitric oxide synthase.

This application claims the benefit of priority of U.S. provisional application No. 60/740,322, filed Nov. 28, 2005, the disclosure of which is hereby incorporated by reference as if written herein in its entirety.

### FIELD OF THE INVENTION

The present invention is directed to salts of compounds that inhibit nitric oxide synthase, their synthesis, and their application as pharmaceuticals for the treatment of disease.

### BACKGROUND OF THE INVENTION

Nitric oxide (NO) is involved in the regulation of many physiological processes as well as the pathophysiology of a number of diseases. It is synthesized enzymatically from L-arginine in numerous tissues and cell types by three distinct isoforms of the enzyme NO synthase (NOS). Two of these isoforms, endothelial NOS (eNOS) and neuronal NOS (nNOS) are expressed in a constitutive manner and are calcium/calmodulin dependent. Endothelial NOS is expressed by endothelium and other cell types and is involved in cardiovascular homeostasis. Neuronal NOS is constitutively present in both the central and peripheral nervous system where NO acts a neurotransmitter. Under normal physiological conditions, these constitutive forms of NOS generate low, transient levels of NO in response to increases in intracellular calcium concentrations. These low levels of NO act to regulate blood pressure, platelet adhesion, gastrointestinal motility, bronchomotor tone and neurotransmission.

In contrast, the third isoform of NOS, inducible NOS (iNOS), a virtually calcium independent enzyme, is absent in resting cells, but is rapidly expressed in virtually all nucleated mammalian cells in response to stimuli such as endotoxins and/or cytokines. The inducible isoform is neither stimulated by calcium nor blocked by calmodulin antagonists. It contains several tightly bound co-factors, including FMN, FAD and tetrahydrobiopterin. The inducible isoform of nitric oxide synthase (NOS₂ or iNOS) is expressed in virtually all nucleated mammalian cells following exposure to inflammatory cytokines or lipopolysaccharide.

The enzyme iNOS synthase is a homodimer composed of 130 kDa subunits. Each subunit comprises an oxygenase domain and a reductase domain. Importantly, dimerization of the iNOS synthase is required for enzyme activity. If the dimerization mechanism is disrupted, the production of nitric oxide via inducible NOS enzyme is inhibited.

The presence of iNOS in macrophages and lung epithelial cells is significant. Once present, iNOS synthesizes 100-1000 times more NO than the constitutive enzymes synthesize and does so for prolonged periods. This excessive production of NO and resulting NO-derived metabolites (e.g., peroxynitrite) elicit cellular toxicity and tissue damage which contribute to the pathophysiology of a number of diseases, disorders and conditions.

Nitric oxide generated by the inducible form of NOS has also been implicated in the pathogenesis of inflammatory diseases. In experimental animals, hypotension induced by lipopolysaccharide or tumor necrosis factor alpha can be reversed by NOS inhibitors. Conditions which lead to cytokine-induced hypotension include septic shock, hemodialysis and interleukin therapy in cancer patients. An iNOS inhibitor has been shown to be effective in treating cytokine-induced hypotension, inflammatory bowel disease, cerebral ischemia, osteoarthritis, asthma and neuropathies such as diabetic neuropathy and post-herpetic neuralgia.

In addition, nitric oxide localized in high amounts in inflamed tissues has been shown to induce pain locally and to enhance central as well as peripheral stimuli. Because nitric oxide produced by an inflammatory response is thought to be synthesized by iNOS, the inhibition of iNOS dimerization produces both prophylactic and remedial analgesia in patients.

Hence, in situations where the overproduction of nitric oxide is deleterious, it would be advantageous to find a specific inhibitor of iNOS to reduce the production of NO. However, given the important physiological roles played by the constitutive NOS isoforms, it is essential that the inhibition of iNOS has the least possible effect on the activity of eNOS and nNOS.

### SUMMARY OF THE INVENTION

Novel salts of compounds, and pharmaceutical compositions thereof that inhibit dimerization of the inducible NOS synthase monomers have been identified, together with methods of synthesizing and using the salts including methods for inhibiting or modulating nitric oxide synthesis and/or lowering nitric oxide levels in a patient by administering the salts.

The salts are formed from a compound of any of the following structural formulas, which are described in U.S. Application Publication No. US2005/0116515A1, the content of which is hereby incorporated by reference in its entirety.

In one aspect, the invention provides salts of compounds of the Formula I:

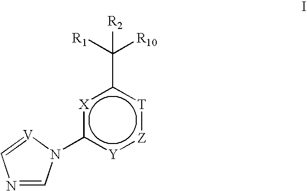

wherein:

T, V, X, and Y are independently selected from the group consisting of CR⁴ and N;

Z is selected from the group consisting of CR³ and N;

R¹ and R² are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally substituted heteroaralkyl, optionally substituted alkene, optionally substituted alkyne, —(O)N(R¹¹)R¹², P(O)[N(R¹¹)R¹²]₂, —SO₂NHC(O)R¹¹, —N(R¹¹)SO₂R¹², —SO₂N(R¹¹)R¹², —NSO₂N(R¹¹)R¹², —C(O)NHSO₂R¹¹, CH═NOR¹¹, —OR¹¹, S(O)ₜ R¹¹, N(R¹¹)R¹², N(R¹¹)C(O)N(R¹²)R¹³, N(R¹¹)C(O)OR¹², N(R¹¹)C(O)R¹², [C(R¹⁴)R¹⁵]Rᵣ—R¹², —[C(R¹⁴)R¹⁵]ᵣ—C(O)OR¹¹, —[C(R¹⁴)R¹⁵]ᵣ—[C(O)OR¹¹]₂, —[C(R¹⁴)R¹⁵]ᵣC(O)N(R¹¹)R¹², —[C(R¹⁴)R¹⁵]ᵣ—N(R¹¹)R¹², —[C(R¹⁴)R¹⁵]ᵣ—N(R¹¹)—[C(R¹⁴) R¹⁵]ᵣ R¹², —[C(R¹⁴)R¹⁵]ᵣ—OR¹¹, —N(R¹¹)—[C(R¹⁴)R¹⁵]ᵣ—R¹², —N(R¹¹)C(O)N(R¹³)—[C(R¹⁴)R¹⁵]ᵣR¹², —C(O)—[C(R¹⁴)R¹⁵]ᵣ—N(R¹¹)R¹², —N(R¹³)C(O)-L-(R¹¹)R¹², —N(R¹¹)—[C(R¹⁴)R¹⁵]ᵣ-L-R¹², —N(R¹¹)C(O)N(R¹¹)—[C(R¹⁴)R¹⁵]ᵣ-L-R¹², —[C(R¹⁴)R¹⁵]ᵣ-L-R¹², and -L-C(O)N(R¹¹)R¹²;

t is an integer from 0 to 2;

r is an integer from 0 to 5;

L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group;

R³ , R⁴, R¹⁰, R¹⁴, R¹⁵, R¹⁶, R¹⁷, and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;

R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)N(R¹⁷)R¹⁸, [C(R¹⁴)R¹⁵]ᵣ—N(R¹⁷)C(O)OR¹⁸, —[C(R¹⁴)R¹⁵]ᵣ—R¹⁷, and —[C(R¹⁴)R¹⁵]ᵣ—N(R¹⁷)C(O)R¹⁸; or R¹¹ or R¹² may be defined by a structure selected from the group consisting of

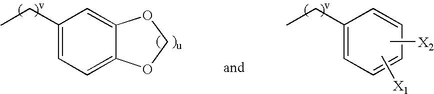

wherein:

u and v are independently an integer from 0 to 3; and

X¹ and X² are selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The invention further provides salts of compounds of the Formula II:

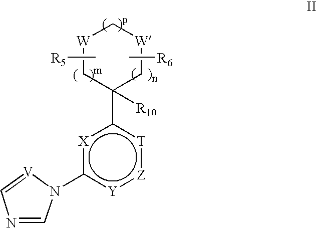

wherein:

T, V, X, and Y are independently selected from the group consisting of CR⁴ and N;

Z is from the group consisting of CR³ and N;

W and W′ are independently selected from the group consisting of CH₂, CR⁷R⁸, NR⁹, O, N(O), S(O)_{q} and C(O);

n, m and p are independently an integer from 0 to 5;

q is 0, 1, or 2;

R³, R⁴, R¹⁰, R¹⁴, R¹⁵, R¹⁶, R¹⁷ and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;

R⁵, R⁶, R⁷, R⁸, and R⁹ are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally optionally substituted heteroaralkyl, optionally substituted alkene, optionally substituted alkyne, —(O)N(R¹¹)R¹², —P(O)[N(R¹¹)R¹²]₂, —SO₂NHC(O)R¹¹, N(R¹¹)SO₂R¹², —SO₂N(R¹¹)R¹², —NSO₂N(R¹¹)R¹², —C(O)NHSO₂R¹¹, CH═NOR¹¹, —OR¹¹, —S(O)ₜ—R¹¹, —N(R¹¹)R¹², —N(R¹¹)C(O)N(R¹²)R¹³, —N(R¹¹)C(O)OR¹², —N(R¹¹)C(O)R¹², —[C(R¹⁴)R¹⁵]ᵣ—R¹², —[C(R¹⁴)R¹⁵]ᵣ—C(O)OR¹¹, —[C(R¹⁴)R¹⁵]ᵣ—[C(O)OR¹¹]₂, [C(R¹⁴)R¹²]ᵣC(O)N(R¹¹)R¹², [C(R¹⁴)R¹⁵]ᵣ N(R¹¹)R¹², [C(R¹⁴)R¹⁵]ᵣ N(R¹¹) [C(R¹⁴)R¹⁵]ᵣ R¹², —[C(R¹⁴)R¹⁵]ᵣ—OR¹¹, —N(R¹¹)—[C(R¹⁴)R¹⁵]ᵣ—R¹², —N(R¹¹)C(O)N(R¹³)—[C(R¹⁴)R¹⁵]ᵣ—R¹², —C(O)—[C(R¹⁴ )R¹⁵]ᵣ—N(R¹¹)R¹², —N(R¹³)C(O)-L-(R¹¹)R¹², —N(R¹¹)—[C(R¹⁴)R¹⁵]ᵣ-L-R¹², —N(R¹¹)C(O)(R¹¹)—[C(R¹⁴)R¹⁵]ᵣ-L-R¹², —[C(R¹⁴)R¹⁵]ᵣ-L-R¹², and -L-C(O)N(R¹¹)R¹²; or R⁵and R⁶ together may form an optionally substituted aryl, optionally substituted heteroaryl, optionally substituted cycloalkyl, or optionally substituted heterocycloalkyl;

t is an integer from 0 to 2;

r is an integer from 0 to 5;

L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group;

R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)N(R¹⁷)R¹⁸, —[C(R¹⁴)R¹⁵]ᵣ—N(R¹⁷)C(O)OR¹⁸, —[C(R¹⁴)R¹⁵]ᵣ—R¹⁷, and —[C(R¹⁴)R¹⁵]ᵣ—N(R¹⁷)C(O)R¹⁸; or R¹¹ or R¹² may be defined by a structure selected from the group consisting of

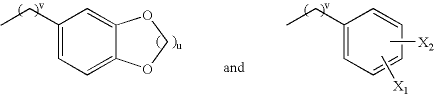

wherein:

u and v are independently an integer from 0 to 3; and

X¹ and X² are independently selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The invention further provides salt of compounds of the Formula III:

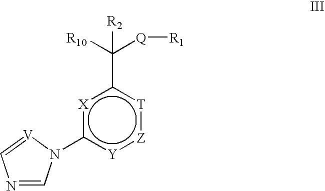

wherein:

V, T, X, and Y are independently selected from the group consisting of CR⁴ and N;

Q is selected from the group consisting of NR⁵, O, and S;

Z is selected from the group consisting of CR³ and N;

R¹ and R² are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally optionally substituted heteroaralkyl, optionally substituted alkene, optionally substituted alkyne, —(O)N(R¹¹)R¹², —P(O)[N(R¹¹)R¹²]₂, —SO₂NHC(O)R¹¹, —N(R¹¹)SO₂R¹², —SO₂N(R¹¹)R¹², —NSO₂N(R¹¹)R¹², —C(O)NHSO₂R¹¹, CH═NOR¹¹, —OR¹¹, —S(O)ₜ—R¹¹, —N(R¹¹)R¹², —N(R¹¹)C(O)N(R¹²)R¹³, —N(R¹¹)C(O)OR¹², —N(R¹¹)C(O)R¹², —[C(R¹⁴)R¹⁵]ᵣ—R¹², —[C(R¹⁴)R¹⁵]ᵣ—C(O)OR¹, —[C(R¹⁴)R¹⁵]ᵣ—[C(O)OR¹¹]₂, —[C(R¹⁴)R¹⁵]ᵣC(O)N(R¹¹)R¹², —[C(R¹⁴)R¹⁵]ᵣ—N(R¹¹)R¹², —[C(R¹⁴)R¹⁵]ᵣ—N(R¹¹)—[C(R¹⁴)R¹⁵]ᵣ R¹², —[C(R¹⁴)R¹⁵]ᵣOR¹¹, —N(R¹¹)—[C(R¹⁴)R¹⁵]ᵣ—R¹², —N(R¹¹)C(O)N(R¹³)—[C(R¹⁴)R¹⁵]ᵣ—R¹², —C(O)—[C(R¹⁴)R¹⁵]ᵣ—N(R¹¹)R¹², —N(R¹³)C(O)-L-(R¹¹)R¹², —N(R¹¹)—[C(R¹⁴)R¹⁵]ᵣL-R¹², —N(R¹¹)C(O)N(R¹¹)—[C(R¹⁴)R¹⁵]ᵣ-L-R¹², [C(R¹⁴)R¹⁵]ᵣ-L-R¹², and -L-C(O)N(R¹¹)R¹²; or R⁵and R⁶ together may form an optionally substituted aryl, optionally substituted heteroaryl, optionally substituted cycloalkyl, or optionally substituted heterocycloalkyl;

t is an integer from 0 to 2;

r is an integer from 0 to 5;

L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group;

R³, R⁴, R¹⁰, R¹⁴, R¹⁵, R¹⁶, R¹⁷, and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;

R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)N(R¹⁷)R¹⁸, —[C(R¹⁴)R¹⁵]ᵣ—N(R¹⁷)C(O)OR¹⁸, —[C(R¹⁴)R⁵]ᵣ—R¹⁷, and —[C(R¹⁴)R¹⁵]ᵣ—N(R¹⁷)C(O)R¹⁸; or R¹¹ or R¹² may be defined by a structure selected from the group consisting of

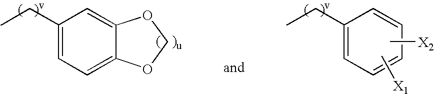

wherein:

u and v are independently an integer from 0 to 3; and

X¹ and X² are independently selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The invention further provides salts of compounds of the Formula IV:

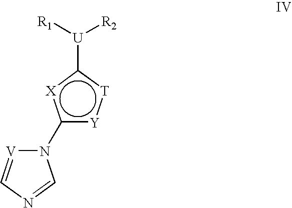

wherein:

T, X, and Y are independently selected from the group consisting of CR⁴, N, NR⁴, S, and O;

U is CR¹⁰ or N;

V is CR⁴ or N;

R¹ and R² are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally optionally substituted heteroaralkyl, optionally substituted alkene, optionally substituted alkyne, —(O)N(R¹¹)R¹², —P(O)[N(R¹¹)R¹²]₂, —SO₂NHC(O)R¹¹, —N(R¹¹)SO₂R¹², —SO₂N(R¹¹)R¹², —NSO₂N(R¹¹)R¹², —C(O)NHSO₂R¹¹, —CH═NOR¹¹, —OR¹¹, —S(O)ₜ—R¹¹, —N(R¹¹)R¹², —N(R¹¹)C(O)N(R¹²)R¹³, —N(R¹¹)C(O)OR¹², —N(R¹¹)C(O)R¹², —[C(R¹⁴)R¹⁵]ᵣ—R¹², —[C(R¹⁴)R¹⁵]ᵣ—C(O)OR¹¹, —[C(R¹⁴)R¹⁵]ᵣ—[C(O)OR¹¹]₂, —[C(R¹⁴)R¹⁵]ᵣC(O)N(R¹¹)R¹², —[C(R¹⁴)R¹⁵]ᵣ—N(R¹¹)R¹², —[C(R¹⁴)R¹⁵]ᵣ—N(R¹¹)—[C(R¹⁴)R¹⁵]ᵣ R¹², —[C(R¹⁴)R¹⁵]ᵣ—N(R¹¹)—C(O)N(R¹¹)R¹², —[C(R¹⁴)R¹⁵]ᵣN(R¹¹)S(O)ₜ—C(O)N(R¹¹)R¹², —[C(R¹⁴)R¹⁵ᵣ—OR¹¹, —N(R¹¹)—[C(R¹⁴)R¹⁵]ᵣ—R¹², —N(R¹¹)C(O)N(R¹³)—[C(R¹⁴)R¹⁵]ᵣ—R¹², —C(O)—[C(R¹⁴)R¹⁵]ᵣ—N(R¹¹)R¹², —N(R¹³)C(O)-L-(R¹¹)R¹², —N(R¹¹)—[C(R¹⁴)R¹⁵]ᵣ-L-R¹², —N(R¹¹)C(O)N(R¹¹)—[C(R¹⁴)R¹⁵]ᵣ-L-R¹², —[C(R¹⁴)R¹⁵]ᵣ-L-R¹², and -L-C(O)N(R¹¹)R¹²; or R⁵ and R⁶ together may form an optionally substituted aryl, optionally substituted heteroaryl, optionally substituted cycloalkyl, or optionally substituted heterocycloalkyl;

t is an integer from 0 to 2;

r is an integer from 0 to 5;

L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group;

R⁴, R¹⁰, R¹⁴, R¹⁵, R¹⁶, R¹⁷, and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;

R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)N(R¹⁷)R¹⁸, —[C(R¹⁴)R¹⁵]ᵣ—N(R¹⁷)C(O)OR¹⁸, —[C(R¹⁴)R¹⁵]ᵣ—R¹⁷, and —[C(R¹⁴)R¹⁵]ᵣ—N(R¹⁷)C(O)R¹⁸; or R¹¹ or R¹² may be defined by a structure selected from the group consisting of

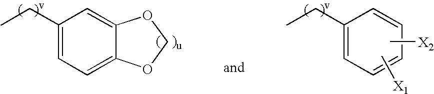

wherein:

u and v are independently an integer from 0 to 3; and

X¹ and X² are independently selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The invention further provides salts of compounds of the Formula V:

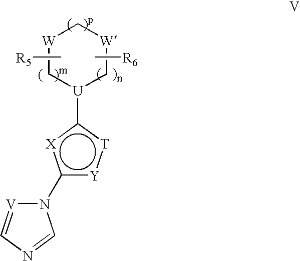

wherein:

T, X, and Y are independently selected from the group consisting of CR⁴, N, NR⁴, S, and O;

U is selected from the group consisting of CR¹⁰ and N;

V is selected from the group consisting of CR⁴ and N;

W and W′ are independently selected from the group consisting of CH₂, CR⁷R⁸, NR⁹, O, N(O), S(O)_{q} and C(O);

n, m and p are independently an integer from 0 to 5;

q is 0, 1, or 2;

R³, R⁴, R₁₀, R¹⁴, R¹⁵, R¹⁶, R¹⁷ and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;

R⁵, R⁶, R⁷, R⁸, and R⁹ are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally optionally substituted heteroaralkyl, optionally substituted alkene, optionally substituted alkyne, —C(O)N(R¹¹)R¹², —P(O)[N(R¹¹)R¹²]₂, —SO₂NHC(O)R¹¹, —N(R¹¹)SO₂R¹², —SO₂N(R¹¹)R¹², —NSO₂N(R¹¹)R¹², —C(O)NHSO₂R¹¹, —CH═NOR¹¹, —OR¹¹, —S(O)ₜ—R¹¹, —N(R¹¹)R¹², —N(R¹¹)C(O)N(R¹²)R¹³, —N(R¹¹)C(O)ORR¹², N(R¹¹)C(O)R¹², —[C(R¹⁴)R¹⁵]ᵣR¹², [C(R¹⁴)R¹⁵]ᵣC(O)OR¹¹, [C(R¹⁴)R¹⁵]ᵣ—[C(O)OR¹¹]₂, —[C(R¹⁴)R¹⁵]ᵣC(O)N(R¹¹)R¹², —[C(R¹⁴)R¹⁵]ᵣ—N(R¹¹)R¹², —[C(R¹⁴)R¹⁵]ᵣ—N(R¹¹)—[C(R¹⁴)R¹⁵]ᵣ R¹², [C(R¹⁴)R¹⁵]ᵣ OR¹¹, N(R¹¹)—[C(R¹⁴)R¹⁵]ᵣ—R¹², N(R¹¹)C(O)N(R¹³)—[C(R¹⁴)R¹⁵]ᵣ R¹², [C(R¹⁴)R¹⁵]ᵣ N(R¹³)—C(O)N(R¹¹)R¹², [C(R¹⁴)R¹⁵]ᵣ—N(R¹³)S(O)ₜ—C(O)N(R¹¹)R¹², —C(O)—[C(R¹⁴)R¹⁵]ᵣ—N(R¹¹)R¹², —N(R¹³)C(O)-L-(R¹¹)R¹², —N(R¹¹)—[C(R¹⁴)R¹⁵]ᵣ-L-R¹², —N(R¹¹)C(O)N(R¹¹)—[C(R¹⁴)R¹⁵]ᵣ-L-R¹², —[C(R¹⁴)R¹⁵]ᵣ-L-R¹², and -L-C(O)N(R¹¹)R¹²; or R⁵ and R⁶ together may form an optionally substituted aryl, optionally substituted heteroaryl, optionally substituted cycloalkyl, or optionally substituted heterocycloalkyl;

t is an integer from 0 to 2;

r is an integer from 0 to 5;

L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group; and

R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)N(R¹⁷)R¹⁸, —[C(R¹⁴)R¹⁵]ᵣ—N(R¹⁷)C(O)OR¹⁸, —[C(R¹⁴)R¹⁵]ᵣ—R¹⁷, and —[C(R¹⁴)R¹⁵]ᵣ N(R¹⁷)C(O)R¹⁸; or R¹¹ or R¹² may be defined by a structure selected from the group consisting of

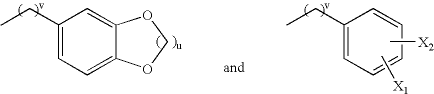

wherein:

u and v are independently an integer from 0 to 3; and

X¹ and X² are independently selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The salts contemplated by the present invention include those salts prepared by combining the compounds of any of Formulas I to V with both acidic and basic reagents. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate,pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of Formulas I to V, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The neutral forms of the compounds may be regenerated by contacting the salt with a base or acid and isolating the parent compound in the conventional manner.

In a broad aspect, the subject invention provides for novel salts, pharmaceutical compositions thereof and methods of making and using the salts and compositions. These salts possess useful nitric oxide synthase inhibiting or modulating activity, and may be used in the treatment or prophylaxis of a disease or condition in which the synthesis or over-synthesis of nitric oxide forms a contributory part. These salts can inhibit and/or modulate the inducible isoform of nitric oxide synthase over the constitutive isoforms of nitric oxide synthase.

### BRIEF DESCRIPTION OF THE DRAWINGS

FIG. 1 shows the XRPD diffraction spectrum for Compound 1, isolated as the hydrochloride (top spectrum) and hydrobromide (middle and bottom spectra) salts. Degrees θ-2θ on the abscissa are plotted against an arbitrary Y value on the ordinate.

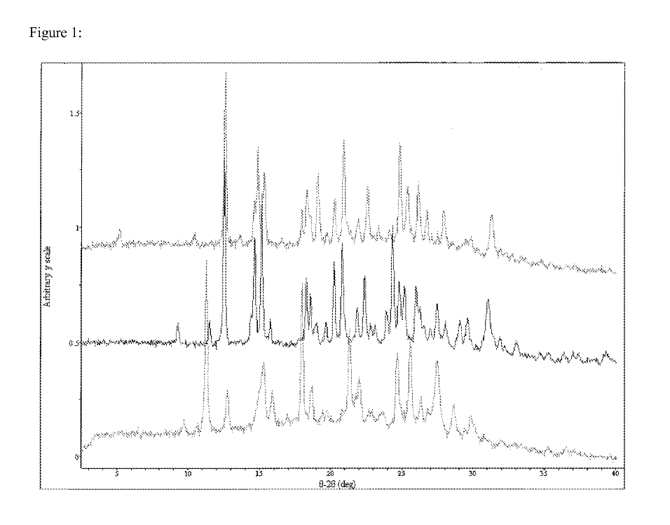

FIG. 2 shows the XRPD diffraction spectrum for Compound 2, isolated as the hydrochloride salt from the salt microscreen (plate-format experiment, bottom spectrum) and from each of the three scale-up attempts (top three spectra). Degrees θ-2θ on the abscissa are plotted against an arbitrary Y value on the ordinate.

### DETAILED DESCRIPTION OF THE INVENTION

Several related broad classes of compounds, disclosed above, may be used in the formation of the salts of the present invention. The present invention also contemplates several preferred embodiments of compounds to be used in the formation of said salts.

In certain embodiments, said compounds are of Formula II wherein Z is CR³ and Y is N.

In certain embodiments, said compounds are of Formula II wherein T is CR⁴.

In certain embodiments, said compounds are of Formula II wherein X is N.

In certain embodiments, said compounds are of Formula II wherein X is CR⁴.

In certain embodiments, said compounds are of Formula II wherein T is N.

In certain embodiments, said compounds are of Formula II wherein X is N.

In certain embodiments, said compounds are of Formula II wherein:

R⁵, R⁶, R⁷, R⁸, and R⁹ are independently selected from the group consisting of hydrogen, halogen, lower alkyl, haloalkyl, optionally substituted aralkyl, optionally substituted aryl, optionally substituted heteroaryl, lower alkene, lower alkyne, (O)N(R¹¹)R¹², P(O)[N(R¹¹)R¹²]₂, SO₂NHC(O)R¹¹, N(R¹¹)SO₂R¹², —SO₂N(R¹¹)H, —C(O)NHSO₂R¹¹, —CH═NOR¹¹, —OR¹¹, —S(O)ₜ—R¹¹, —N(R¹¹)R¹², —N(R¹¹)C(O)N(R¹²)R¹³, —N(R¹¹)C(O)OR¹², —N(R¹¹)C(O)R¹², —[C(R¹⁴)R¹⁵]ᵣ—C(O)OR¹¹, —[C(R¹⁴)R¹⁵]ᵣ—[C(O)OR¹¹]₂, —[C(R¹⁴)R¹⁵]ᵣ—N(R¹¹)R¹², —[C(R¹⁴)R¹⁵]ᵣC(O)N(R¹¹)R¹², —N(R¹¹)—[C(R¹⁴)R¹⁵]ᵣ—R¹², —N(R¹¹)C(O)N(R¹²)—[C(R¹⁴)R¹⁵]ᵣ—R¹², —[C(R¹⁴)R¹⁵]ᵣ—R¹², —N(R¹¹)—[C(R¹⁴)R¹⁵]ᵣ-L-R¹², —[C(R¹⁴)R¹⁵]ᵣ-L-R¹² and —N(R¹¹)C(O)N(R¹²)R¹³—[C(R¹⁴)R¹⁵]ᵣ-L-R¹²; or R⁵ and R⁶ together may form an optionally substituted aryl, optionally substituted heteroaryl, optionally substituted cycloalkyl, or optionally substituted heterocycloalkyl;

R³, R⁴, R¹⁰, R¹⁴, R¹⁵, R¹⁶, R¹⁷ and R¹⁸ are independently selected from the group consisting of hydrogen, halogen, lower alkyl, haloalkyl, optionally substituted aralkyl, optionally substituted aryl, optionally substituted heteroaryl, lower alkene, and lower alkyne; or R¹⁴ and R¹⁵ may together form a carbonyl, optionally substituted carbocycle or optionally substituted heterocycle; and

R¹¹, R¹², and R¹³ are independently selected from the group consisting of hydrogen, halo, lower alkyl, haloalkyl, optionally substituted aralkyl, optionally substituted aryl, optionally substituted heteroaralkyl, optionally substituted heteroaryl, lower alkene, and lower alkyne; or R¹¹ or R¹² may be defined by a structure selected from the group consisting of

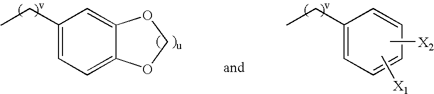

wherein:

u and v are independently an integer from 0 to 3; and

X¹ and X² are independently selected from the group consisting of hydrogen, halogen, hydroxy, lower acyloxy, lower alkyl, lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

In certain embodiments, the invention further provides for compounds of Formula II wherein:

R⁷, R⁸, and R⁹ are independently selected from the group consisting of hydrogen, halogen, lower alkyl, haloalkyl, optionally substituted aralkyl, optionally substituted aryl, optionally substituted heteroaryl, lower alkene, lower alkyne, —N(R¹¹)SO₂R¹², —SO₂N(R¹¹)H, —OR¹¹, —S(O)ₜ—R¹¹, —N(R¹¹)R¹², N(R¹¹)C(O)N(R¹²)R¹³, N(R¹¹)C(O)R¹², —[C(R¹⁴)R¹⁵]ᵣN(R¹¹)R¹², —[C(R¹⁴)R¹⁵]ᵣ—C(O)N(R¹¹)R¹², —N(R¹¹)—[C(R¹⁴)R¹⁵]ᵣ—R¹², —N(R¹¹)—[C(R¹⁴)R¹⁵]ᵣ-L-R¹², —[C(R¹⁴)R¹⁵]ᵣ-L-R¹² and —N(R¹¹)C(O)N(R¹²)R¹³—[C(R¹⁴)R¹⁵]ᵣ-L-R¹²; and

R⁵ and R⁶ are independently selected from the group consisting of hydrogen, halo, lower alkyl, haloalkyl, optionally substituted aralkyl, optionally substituted aryl, optionally substituted heteroaryl, lower alkene, lower alkyne, —N(R¹¹)C(O)R¹², —[C(R¹⁴)R¹⁵]ᵣ—C(O)OR¹¹, —[C(R¹⁴)R¹⁵]ᵣ—N(R¹¹)R¹², —[C(R¹⁴)R¹⁵]ᵣ—C(O)N(R¹¹)R¹², and —N(R¹¹)—[C(R¹⁴)R¹⁵]ᵣ—R¹², or R⁵ and R⁶ together may form an optionally substituted aryl, optionally substituted heteroaryl, optionally substituted cycloalkyl, or optionally substituted heterocycloalkyl.

In certain embodiments, said compounds are of Formula II wherein R⁷ or R⁹ is independently selected from the group consisting of hydrogen, halogen, lower alkyl, haloalkyl, optionally substituted aralkyl, optionally substituted aryl, optionally substituted heteroaryl, lower alkene, lower alkyne, —N(R¹¹)SO₂R¹², —SO₂N(R¹¹)H, —OR¹¹, —S(O)ₜ—R¹¹, —N(R¹¹)R¹², —N(R¹¹)C(O)N(R¹²)R¹³, —N(R¹¹)C(O)R¹², [C(R¹⁴)R¹⁵]ᵣ N(R¹¹)R¹², [C(R¹⁴)R¹⁵]ᵣ C(O)N(R¹¹)R¹², and N(R¹¹)[C(R¹⁴)R¹⁵]ᵣ R¹².

In certain embodiments, said compounds are of Formula II wherein W is CH₂ and W′ is NR⁹. The invention further provides for compounds of Formula II wherein m, n, and p are each independently an integer from 0 to 2. The invention further provides for compounds of Formula II wherein R⁹ is selected from the group consisting of —C(O)N(R¹¹)R¹² and —[C(R¹⁴)R¹⁵]ᵣ—N(R¹¹)R¹². The invention yet further provides for compounds of Formula II wherein R⁹ is —[C(R¹⁴)R¹⁵]ᵣ—N(R¹¹)R¹². The invention yet further provides for compounds of Formula II wherein r is 2.

In certain embodiments, said compounds are of Formula II wherein R¹¹ is selected from the group consisting of hydrogen and lower alkyl. In further embodiments, said compounds are of Formula II wherein R¹¹ is selected from the group consisting of hydrogen and methyl. In yet further embodiments, said compounds are of Formula II wherein wherein R¹¹ is hydrogen.

In certain embodiments, said compounds are of Formula II wherein R¹² is defined by the following structural formula: 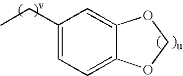
wherein u and v are independently an integer from 0 to 3. In further embodiments, said compounds are of Formula II wherein u and v are independently 1 or 2.

In certain embodiments, said compounds are of Formula II wherein p and m are 1 and n is 0.

In certain embodiments, said compounds are of Formula II wherein R¹⁴ and R¹⁵ are hydrogen.

In certain embodiments, said compounds are of Formula II wherein R⁴, R⁵, R⁶ and R¹⁰ are hydrogen.

In certain embodiments, said compounds are of Formula II wherein R³ is methyl.

In certain embodiments, said compounds are of Formula II wherein u and v are each 1.

In certain embodiments, said compounds are of Formula II wherein T is CR⁴ and X is N.

In certain embodiments, said compounds are of Formula IV wherein T and X are independently selected from the group consisting of CR⁴ and N, and Y is selected from the group consisting of S and O.

In certain embodiments, said compounds are of Formula IV wherein T is selected from the group consisting of S and O, and X and Y are selected from the group consisting of CR⁴ and N.

In certain embodiments, said compounds are of Formula IV wherein Y is N.

In certain embodiments, said compounds are of Formula IV wherein X is N.

In certain embodiments, said compounds are of Formula IV wherein T is S.

In certain embodiments, said compounds are of Formula IV wherein V is CR⁴.

In certain embodiments, said compounds are of Formula IV wherein:

R¹ and R² are independently selected from the group consisting of hydrogen, halogen, lower alkyl, haloalkyl, optionally substituted aralkyl, optionally substituted aryl, optionally substituted heteroaryl, lower alkene, lower alkyne, —(O)N(R¹¹)R¹², —P(O)[N(R¹¹)R¹²]₂, —SO₂NHC(O)R¹¹, —N(R¹¹)SO₂R¹², —SO₂N(R¹¹)H, —C(O)NHSO₂R¹¹, —CH═NOR¹¹, —OR¹¹, —S(O)ₜ—R¹¹, —N(R¹¹)R¹², —N(R¹¹)C(O)N(R¹²)R¹³, N(R¹¹)C(O)OR¹², N(R¹¹)C(O)R¹², [C(R¹⁴)R¹⁵]ᵣ C(O)OR¹¹, [C(R¹⁴)R¹⁵]ᵣ [C(O)OR¹¹]₂, —[C(R¹⁴)R¹⁵]ᵣ—N(R¹¹)R¹², —[C(R¹⁴)R¹⁵]ᵣC(O)N(R¹¹)R¹², —N(R¹¹)—[C(R¹⁴)R¹⁵]ᵣ—R¹², —N(R¹¹)C(O)N(R¹²)—[C(R¹⁴)R¹⁵]ᵣ—R¹², —[C(R¹⁴)R¹⁵]ᵣ—R¹², —[C(R¹⁴)R¹⁵]ᵣ—N(R¹³)—C(O)N(R¹¹)R¹², —[C(R¹⁴)R¹⁵]ᵣ—N(R¹¹)S(O)ₜ—C(O)N(R¹¹)R¹², —N(R¹¹)—[C(R¹⁴)R¹⁵]ᵣ-L-R¹², —[C(R¹⁴)R¹⁵]ᵣ-L-R¹² and —N(R¹¹)C(O)N(R¹²)R¹³—[C(R¹⁴)R¹⁵]ᵣ-L-R¹²; or R⁵ and R⁶ together may form an optionally substituted aryl, optionally substituted heteroaryl, optionally substituted cycloalkyl, or optionally substituted heterocycloalkyl;

R⁴, R¹⁰, R¹⁴, R¹⁵, R¹⁶, R¹⁷ and R¹⁸ are independently selected from the group consisting of hydrogen, halogen, lower alkyl, haloalkyl, optionally substituted aralkyl, optionally substituted aryl, optionally substituted heteroaryl, lower alkene, and lower alkyne; or R¹⁴ and R¹⁵ may together form a carbonyl, optionally substituted carbocycle or optionally substituted heterocycle; and

R¹¹, R¹², and R¹³ are independently selected from the group consisting of hydrogen, halo, lower alkyl, haloalkyl, optionally substituted aralkyl, optionally substituted aryl, optionally substituted heteroaralkyl, optionally substituted heteroaryl, lower alkene, and lower alkyne; or R¹¹ or R¹² may be defined by a structure selected from the group consisting of

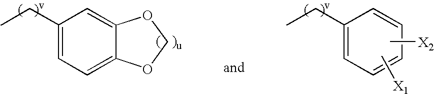

wherein:

u and v are independently an integer from 0 to 3; and

X¹ and X² are independently selected from the group consisting of hydrogen, halogen, hydroxy, lower acyloxy, lower alkyl, lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The invention further provides for compounds of Formula IV wherein:

R¹ is selected from the group consisting of hydrogen, halogen, lower alkyl, haloalkyl, optionally substituted aralkyl, optionally substituted aryl, optionally substituted heteroaryl, lower alkene, lower alkyne, —N(R¹¹)SO₂R¹², —SO₂N(R¹¹)H, —OR¹¹, —S(O)ₜ—R¹¹, N(R¹¹)R¹², —N(R¹¹)C(O)N(R¹²)R¹³, —N(R¹¹)C(O)R¹², —[C(R¹⁴)R¹⁵]ᵣ—N(R¹¹)R¹², —[C(R¹⁴)R¹⁵]ᵣ—C(O)N(R¹¹)R¹², —N(R¹¹)—[C(R¹⁴)R¹⁵]ᵣ—R¹², —N(R¹¹)—[C(R¹⁴)R¹⁵]ᵣ-L-R¹², —[C(R¹⁴)R¹⁵]ᵣ-L-R¹², —N(R¹¹)C(O)N(R¹²)R¹³—[C(R¹⁴)R¹⁵]ᵣ-L-R¹², —[C(R¹⁴)R¹⁵]ᵣ—N(R¹³)—C(O)N(R¹¹)R¹², and —[C(R¹⁴)R¹⁵]ᵣ—N(R¹³)S(O)ₜ—C(O)N(R¹¹)R¹²; and

R² is selected from the group consisting of hydrogen, halo, lower alkyl, haloalkyl, optionally substituted aralkyl, optionally substituted aryl, optionally substituted heteroaryl, lower alkene, lower alkyne, —N(R¹¹)C(O)R¹², —[C(R¹⁴)R¹⁵]ᵣ—C(O)OR¹¹, —[C(R¹⁴)R¹⁵]ᵣ—N(R¹¹)R¹², —[C(R¹⁴)R¹⁵]ᵣ—C(O)N(R¹¹)R¹², and —N(R¹¹)—[C(R¹⁴)R¹⁵]ᵣ—R¹²,

The invention yet further provides for compounds of Formula IV wherein R¹ is selected from the group consisting of hydrogen, halogen, lower alkyl, haloalkyl, optionally substituted aralkyl, optionally substituted aryl, optionally substituted heteroaryl, lower alkene, lower alkyne, —N(R¹¹)SO₂R¹², —SO₂N(R¹¹)H, —OR¹¹, —S(O)ₜ—R¹¹, —N(R¹¹)R¹², —N(R¹¹)C(O)N(R¹²)R¹³, —N(R¹¹)C(O)R¹², —[C(R¹⁴)R¹⁵]ᵣ—N(R¹¹)R¹², —[C(R¹⁴)R¹⁵]ᵣ—C(O)N(R¹¹)R¹², —N(R¹¹)—[C(R¹⁴)R¹⁵]ᵣ—R¹², [C(R¹⁴)R¹⁵]ᵣ—N(R¹³)—C(O)N(R¹¹)R¹², and —[C(R¹⁴)R¹⁵]ᵣ—N(R¹³)S(O)ₜ—C(O)N(R¹¹)R¹².

In certain embodiments, said compounds are of Formula IV wherein U is N.

In certain embodiments, said compounds are of Formula IV wherein R¹ is selected form the group consisting of —[C(R¹⁴)R¹⁵]ᵣ—N(R¹¹)R¹², —[C(R¹⁴)R¹⁵]ᵣ—C(O)N(R¹¹)R¹², —[C(R¹⁴)R¹⁵]ᵣ—N(R¹³)—C(O)N(R¹¹)R¹², and —[C(R¹⁴)R¹⁵]ᵣ—N(R¹³)S(O)ₜ—C(O)N(R¹¹)R¹².

In certain embodiments, said compounds are of Formula IV wherein R¹² is selected from the group consisting of NH₂ and heteroaryl, or is defined by one of the following structural formulae:

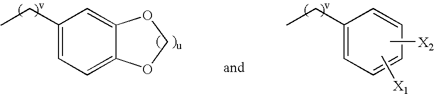

wherein:

u and v are independently an integer from 0 to 3; and

X¹ and X² are selected from the group consisting of hydrogen, halogen, hydroxy, lower acyloxy, lower alkyl, lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

In further embodiments, said compounds are of Formula IV wherein wherein X₁ and X₂ are independently selected from the group consisting of hydrogen, halogen, hydroxy, lower alkyl, lower alkoxy, lower haloalkyl, lower haloalkoxy, and lower perhaloalkyl.

In certain embodiments, said compounds are of Formula IV wherein R⁹ is —[C(R¹⁴)R¹⁵]ᵣ—N(R¹¹)R¹².

In certain embodiments, said compounds are of Formula IV wherein R¹² is defined by the following structural formula:

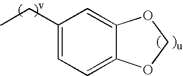

and u and v are independently 1 or 2.

In certain embodiments, said compounds are of Formula IV wherein R¹⁴ and R¹⁵ are both hydrogen.

In certain embodiments, said compounds are of Formula IV wherein R² is selected from the group consisting of hydrogen and lower alkyl.

In certain embodiments, said compounds are of Formula IV wherein R¹¹ is hydrogen or methyl.

In certain embodiments, said compounds are of Formula IV wherein R² is methyl.

In certain embodiments, said compounds are of Formula IV wherein R¹⁰, R¹¹, and R⁴ are hydrogen, and u and v are 1.

In certain embodiments, said compounds are of Formula IV wherein Y and X are N, T is S, and V is CR⁴.

In certain embodiments, said compounds are of Formula IV wherein T and X are independently selected from the group consisting of CR⁴ and N, and Y is selected from the group consisting of S and O.

In certain embodiments, said compounds are of Formula IV wherein T is selected from the group consisting of S and O, and X and Y are independently selected from the group consisting of CR⁴ and N.

In certain embodiments, said compounds are of Formula V wherein Y is N.

In certain embodiments, said compounds are of Formula V wherein X is N.

In certain embodiments, said compounds are of Formula V wherein T is S.

In certain embodiments, said compounds are of Formula V wherein V is CR⁴.

In certain embodiments, said compounds are of Formula V wherein Y is CR⁴.

In certain embodiments, said compounds are of Formula V wherein:

R⁵, R⁶, R⁷, R⁸, and R⁹ are independently selected from the group consisting of hydrogen, halogen, lower alkyl, haloalkyl, optionally substituted aralkyl, optionally substituted aryl, optionally substituted heteroaryl, lower alkene, lower alkyne, —C(O)N(R¹¹)R¹², —P(O)[N(R¹¹)R¹²]₂, —SO₂NHC(O)R¹¹, —N(R¹¹)SO₂R¹², —SO₂N(R¹¹)H, —C(O)NHSO₂R¹¹, —CH═NOR¹¹, —OR¹¹, —S(O)ₜ—R¹¹, —N(R¹¹)R¹², —N(R¹¹)C(O)N(R¹²)R¹³, —N(R¹¹)C(O)OR¹², N(R¹¹)C(O)R¹², —[C(R⁴)R¹⁵]ᵣ—C(O)OR¹¹, —[C(R¹⁴)R¹⁵]ᵣ—[C(O)OR¹¹]₂, —[C(R¹⁴)R¹⁵]ᵣ—N(R¹¹)R¹², —[C(R¹⁴)R¹⁵]ᵣC(O)N(R¹¹)R¹², —N(R¹¹)—[C(R¹⁴)R¹⁵]ᵣ—R¹², —N(R¹¹)C(O)N(R¹²)—[C(R¹⁴)R¹⁵]ᵣ—R¹², —[C(R¹⁴)R¹⁵]ᵣ—R¹², —N(R¹¹)—[C(R¹¹)—[C(R¹⁴)R⁵]ᵣ-L-R¹², —[C(R¹⁴)R¹⁵]ᵣ-L-R¹² and —N(R¹¹)C(O)N(R¹²)R¹³—[C(R¹⁴)R¹⁵]ᵣ-L-R¹²; or R⁵ and R⁶ together may form an optionally substituted aryl, optionally substituted heteroaryl, optionally substituted cycloalkyl, or optionally substituted heterocycloalkyl;

R³, R⁴, R₁₀, R¹⁴, R¹⁵, R¹⁶, R¹⁷ and R¹⁸ are independently selected from the group consisting of hydrogen, halogen, lower alkyl, haloalkyl, optionally substituted aralkyl, optionally substituted aryl, optionally substituted heteroaryl, lower alkene, and lower alkyne; or R¹⁴ and R¹⁵ may together form a carbonyl, optionally substituted carbocycle or optionally substituted heterocycle; and

R¹¹, R¹², and R¹³ are independently selected from the group consisting of hydrogen, halo, lower alkyl, haloalkyl, optionally substituted aralkyl, optionally substituted aryl, optionally substituted heteroaralkyl, optionally substituted heteroaryl, lower alkene, and lower alkyne; or R¹¹ or R¹² may be defined by a structure selected from the group consisting of

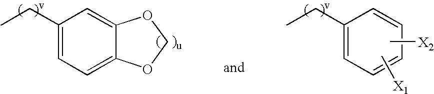

wherein:

u and v are independently an integer from 0 to 3; and

X¹ and X² are independently selected from the group consisting of hydrogen, halogen, hydroxy, lower acyloxy, lower alkyl, lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

The invention further provides for compounds of Formula V wherein:

R⁷, R⁸, and R⁹ are independently selected from the group consisting of hydrogen, halogen, lower alkyl, haloalkyl, optionally substituted aralkyl, optionally substituted aryl, optionally substituted heteroaryl, lower alkene, lower alkyne, —C(O)N(R¹¹)R¹², —[C(R¹⁴)R¹⁵]ᵣ—N(R¹¹)R¹², —N(R¹¹)SO₂R¹², —SO₂N(R¹¹)H, —OR¹¹, —S(O)ₜ—R¹¹, —N(R¹¹)R¹², —N(R¹¹)C(O)N(R¹²)R¹³, —N(R¹¹)C(O)R¹², —[C(R¹⁴)R¹⁵]ᵣ—N(R¹¹)R¹², [C(R¹⁴)R¹⁵]ᵣ C(O)N(R¹¹)R¹², N(R¹¹) [C(R¹⁴)R¹⁵]ᵣ R¹², N(R¹¹) [C(R¹⁴)R¹⁵]ᵣ L R¹², [C(R¹⁴)R¹⁵]ᵣ-L-R¹² and —N(R¹¹)C(O)N(R¹²)R¹³—[C(R¹⁴)R¹⁵]ᵣ-L-R¹²; and

R⁵ and R⁶ are independently selected from the group consisting of hydrogen, halo, lower alkyl, haloalkyl, optionally substituted aralkyl, optionally substituted aryl, optionally substituted heteroaryl, lower alkene, lower alkyne, —OR¹¹, —S(O)ₜ—R¹¹, —N(R¹¹)R¹², —N(R¹¹)C(O)R¹², [C(R¹⁴)R¹⁵]ᵣ—C(O)OR¹¹, —[C(R¹⁴)R¹⁵]ᵣ—N(R¹¹)R¹², [C(R¹⁴)R¹⁵]ᵣ—C(O)N(R¹¹)R¹², and —N(R¹¹)—[C(R¹⁴)R¹⁵]ᵣ—R¹², or R⁵ and R⁶ together may form an optionally substituted aryl, optionally substituted heteroaryl, optionally substituted cycloalkyl, or optionally substituted heterocycloalkyl.

The invention yet further provides for compounds of Formula V wherein R⁷ and R⁹ are independently selected from the group consisting of hydrogen, halogen, lower alkyl, haloalkyl, optionally substituted aralkyl, optionally substituted aryl, optionally substituted heteroaryl, lower alkene, lower alkyne, —N(R¹¹)SO₂R¹², —SO₂N(R¹¹)H, —OR¹¹, —S(O)ₜ—R¹¹, —N(R¹¹)R¹², —N(R¹¹)C(O)R¹²)R¹³, —N(R¹¹)C(O)R¹², —[C(R¹⁴)R¹⁵]ᵣ—N(R¹¹)R¹², —[C(R¹⁴)R¹⁵]ᵣ—C(O)N(R¹¹)R¹², and —N(R¹¹)—[C(R¹⁴)R¹⁵]ᵣ—R¹².

In certain embodiments, said compounds are of Formula V wherein R¹² is defined by the following structural formula:

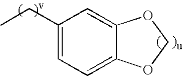

wherein u and v are independently an integer from 0 to 3. The invention further provides for compounds of Formula V wherein u and v are independently 1 or 2.

In certain embodiments, said compounds are of Formula V wherein R¹¹ is selected from the group consisting of hydrogen and lower alkyl. The invention further provides for compounds of Formula V wherein R¹¹ is selected from the group consisting of hydrogen and methyl. The invention yet further provides for compounds of Formula V wherein R³ is methyl.

In certain embodiments, said compounds are of Formula V wherein U is N, W is CH₂, and W′ is CR⁷R⁸.

In certain embodiments, said compounds are of Formula V wherein U is CR⁴, W is CH₂, and W′ is NR⁹.

The invention further provides for compounds of Formula V wherein R⁸ is selected from the group consisting of —C(O)N(R¹¹)R¹² and —[C(R¹⁴)R¹⁵]ᵣ—N(R¹¹)R¹².

In certain embodiments, said compounds are of Formula V wherein R¹⁴ and R¹⁵ are hydrogen.

In certain embodiments, said compounds are of Formula V wherein wherein r is 1 to 3.

In certain embodiments, said compounds are of Formula V wherein R⁷ is hydrogen.

In certain embodiments, said compounds are of Formula V wherein R⁵ is selected from the group consisting of hydrogen, —OR¹¹, —S(O)ₜ—R¹¹, and —N(R¹¹)R¹². In certain embodiments, said compounds are of Formula V wherein R¹¹ is hydrogen or methyl.

In certain embodiments, said compounds are of Formula V wherein R¹² is defined by the following structural formula:

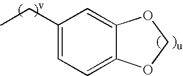

and u and v are independently 1 or 2.

In certain embodiments, said compounds are of Formula V wherein R⁴ and R⁶ and are hydrogen.

Each salt of the invention can be made from a preparation of a compound of any of Formulas I to V. The compounds of any of Formulas I to V can be synthesized or obtained according to any method apparent to those of skill in the art. In preferred embodiments, compounds of any of Formulas I to V are prepared according to the methods described in detail in U.S. Application Publication No. US2005/0116515A1, the content of which is hereby incorporated by reference in its entirety. The compounds of any of Formulas I to V prepared by any method can be contacted with an appropriate acid, either neat or in a suitable inert solvent, to yield the salt forms of the invention.

Several compounds were prepared as various salts, as enumerated in the Examples below, and the present invention provides for these salts. There exist a variety of techniques well-known in the art for preparing salts, and the present invention contemplates these methods without limitation. Two protocols, described below in Examples 8 and 9, were employed in an initial screen of approximately 30 acids for their suitability in preparation of salts.

A number of acids resulted in samples of particular interest as salts suitable to the compounds of the present invention. Thus, in certain embodiments, the present invention provides for a salt of a compound as disclosed herein wherein said salt is selected from the group consisting of acetate, adipate, L-ascorbate, benzenesulfonate (besylate), benzoate, citrate, fumarate, gentisate, glutarate, glycolate, hippurate, hydrochloride, hydrobromide, 1-hydroxy-2-napthoate, p-hydroxybenzoate, maleate, L-malate, malonate, DL mandelate, methanesulfonate (mesylate), nicotinate, oxalate, phosphate, p-toluenesulfonate (tosylate), pyroglutamate, succinate, sulfate, L-(+)tartrate, DL-tartarate, and trifluoroacetate salts. In further embodiments, the salt will be selected from the group consisting of the hydrochloride, hydrobromide, trifluoroacetate, acetate, adipate, p-toluenesulfonate, glycolate, oxalate, fumarate, and phosphonate salts of a compound as disclosed herein. In certain embodiments, particularly preferred salts include hydrochloride, acetate, and adipate salts of a compound as disclosed herein. In further embodiments, most preferred is the acetate salt.

In certain embodiments, the compound is a compound of any of Formulas I to V. In further embodiments, said formula is selected from the group consisting of Formulas II and IV. In yet further embodiments, said formula is Formula II. In yet further embodiments, said compound is Compound 1. In yet further embodiments, said salt is selected from the group consisting of hydrochloride, acetate, adipate, oxalate, phosphate, and hippurate. In other embodiments, said formula is Formula IV. In further embodiments, said compound is Compound 2. In yet further embodiments, said salt is selected from the group consisting of hydrochloride, acetate, and adipate. In yet further embodiments, the salt is the adipate salt of Compound 2.

The present invention also provides for a salt of N-benzo[1,3]dioxol-5-ylmethyl-N-(3-imidazol-1-yl-[1,2,4]thiadiazol-5-yl)-N-methyl-propane-1,3-diamine. The present invention also provides for N′-benzo[1,3]dioxol-5-ylmethyl-N-(3-imidazo-1-yl-[1,2,4]thiadiazol-5-yl)-N-methyl-propane-1,3-diamine acetate. The present invention also provides for N′-benzo[1,3]dioxol-5-ylmethyl-N-(3-imidazol-1-yl-[1,2,4]thiadiazol-5-yl)-N-methyl-propane-1,3-diamine hydrochloride. The present invention also provides for N′-benzo[1,3]dioxol-5-ylmethyl-N-(3-imidazol-1-yl-[1,2,4]thiadiazol-5-yl)-N-methyl-propane-1,3-diamine adipate.

Amongst the salts disclosed herein, a number of properties distinguish the more desirable salts from those that are less desirable. One such property is the readiness with which a salt is formed or purified. Another such property is the stability of a given salt compound over time; that is, its resistance to degradation, oxidation, polymerization, etc. Hygroscopicity is one useful early indicator of compound stability over time. Yet another such property is the solubility of a given salt. Generally, an ideal salt will be readily soluble in a buffer or aqueous solution that mimics plasma or other physiological conditions.

The present invention also provides for a salt of a compound as disclosed herein, formulated for topical administration.

The present invention also provides for a salt of a compound as disclosed herein, for use as a medicament.

The present invention also provides for a salt of a compound as disclosed herein, useful for the treatment or prevention of an iNOS-mediated disease.

The present invention also provides a method for achieving an effect in a patient comprising the administration of a therapeutically effective amount of a salt of a compound as disclosed herein to a patient, wherein the effect is selected from the group consisting of inhibition if iNOS and treatment of an iNOS-mediated disease.

In certain embodiments, said disease is selected from the group consisting of inflammation, inflammatory pain, neuropathic pain, post-herpetic neuralgia, post-surgical pain, and an ocular disease.

The present invention provides for a salt of an iNOS inhibitor.

The present invention provides particular pharmaceutically acceptable salts of compounds of any of Formulas I to V, potent inhibitors of NOS and in particular iNOS, having particular utility for the treatment or prevention of conditions and disorders associated with inflammation and pain.

Salts of the subject invention are useful in treating nitric oxide synthase-mediated disease, disorders and conditions, and are particularly suitable as inhibitors of nitric oxide synthase dimerization. The salts of the present invention are useful to treat patients with neuropathy or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The salts are also useful in the treatment of pain associated with acute herpes zoster (shingles), postherpetic neuralgia (PHN), and associated pain syndromes such as ocular pain. The salts are further useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The salts are also useful for the treatment of pain-related disorders such as tactile allodynia and hyperalgesia. The pain may be somatogenic (either nociceptive or neuropathic), acute and/or chronic. The nitric oxide dimerization inhibitors of the subject invention are also useful in conditions where NSAIDs, morphine or fentanyl opiates and/or other opioid analgesics would traditionally be administered.

Furthermore, the salts of the subject invention can be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the salts and methods of the present invention are useful in the treatment or prevention of drug withdrawal symptoms, for example treatment or prevention of symptoms of withdrawal from opiate, alcohol, or tobacco addiction.

In addition, the salts of the subject invention can be used to treat insulin resistance and other metabolic disorders such as atherosclerosis that are typically associated with an exaggerated inflammatory signaling.

The present invention encompasses therapeutic methods using novel selective iNOS inhibitors to treat or prevent respiratory disease or conditions, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchioectasis cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

The salts of the present invention are also useful in treating inflammation and related conditions. The salts of the present invention are useful as anti-inflammatory agents with the additional benefit of having significantly less harmful side effects. The salts are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The salts are also useful in treating osteoporosis and other related bone disorders. These salts can also be used to treat gastrointestinal conditions such as reflux esophagitis, diarrhea, inflammatory bowel disease, Crohn's disease, gastritis, irritable bowel syndrome and ulcerative colitis. The salts may also be used in the treatment of pulmonary inflammation, such as that associated with viral infections and cystic fibrosis. In addition, salts of invention are also useful in organ transplant patients either alone or in combination with conventional immunomodulators. Yet further, the salts of the invention are useful in the treatment of pruritis and vitaligo.

The salts of the present invention are also useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like.

The salts of the subject invention are also useful for the treatment of certain diseases and disorders of the nervous system. Central nervous system disorders in which nitric oxide inhibition is useful include cortical dementias including Alzheimer's disease, central nervous system damage resulting from stroke, ischemias including cerebral ischemia (both focal ischemia, thrombotic stroke and global ischemia (for example, secondary to cardiac arrest), and trauma. Neurodegenerative disorders in which nitric oxide inhibition is useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia e.g. pre-senile dementia, and AIDS-related dementia, cachexia, Sydenham's chorea, Huntington's disease, Parkinson's Disease, amyotrophic lateral sclerosis (ALS), Korsakoffs disease, imbecility relating to a cerebral vessel disorder, sleeping disorders, schizophrenia, depression, depression or other symptoms associated with Premenstrual Syndrome (PMS), and anxiety.

Furthermore, the salts of the present invention are also useful in inhibiting NO production from L-arginine including systemic hypotension associated with septic and/or toxic hemorrhagic shock induced by a wide variety of agents; therapy with cytokines such as TNF, IL-1 and IL-2; and as an adjuvant to short term immunosuppression in transplant therapy. These salts can also be used to treat allergic rhinitis, respiratory distress syndrome, endotoxin shock syndrome, and atherosclerosis.

Still other disorders or conditions advantageously treated by the salts of the subject invention include the prevention or treatment of cancer, such as colorectal cancer, and cancer of the breast, lung, prostate, bladder, cervix and skin. Salts of the invention may be used in the treatment and prevention of neoplasias including but not limited to brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present salts and methods can also be used to treat the fibrosis which occurs with radiation therapy. The present salts and methods can be used to treat subjects having adenomatous polyps, including those with familial adenomatous polyposis (FAP). Additionally, the present salts and methods can be used to prevent polyps from forming in patients at risk of FAP.

The salts of the subject invention can be used in the treatment of ophthalmic diseases, such as glaucoma, retinal ganglion degeneration, ocular ischemia, retinitis, retinopathies, uveitis, ocular photophobia, and of inflammation and pain associated with acute injury to the eye tissue. Specifically, the salts can be used to treat glaucomatous retinopathy and/or diabetic retinopathy. The salts can also be used to treat post-operative inflammation or pain as from ophthalmic surgery such as cataract surgery and refractive surgery.

Moreover, salts of the subject invention may be used in the treatment of menstrual cramps, dysmenorrhea, premature labor, tendonitis, bursitis, skin-related conditions such as psoriasis, eczema, burns, sunburn, dermatitis, pancreatitis, hepatitis, and the like. Other conditions in which the salts of the subject invention provides an advantage in inhibiting nitric oxide inhibition include diabetes (type I or type II), congestive heart failure, myocarditis, atherosclerosis, and aortic aneurysm.

The present salts may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The salts of the subject invention may also be used to prevent tissue damage when therapeutically combined with antibacterial or antiviral agents.

Besides being useful for human treatment, these salts are also useful for veterinary treatment of companion animals, exotic animals and farm animals, including mammals, rodents, and the like. More preferred animals include horses, dogs, and cats.

While it may be possible for the salts of the subject invention to be administered as the raw chemical, it is also possible to present them as a pharmaceutical formulation. Accordingly, the subject invention provides a pharmaceutical formulation comprising a salt of a compound of any of Formulas I-V, or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a salt of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, dectuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered salt moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active salts may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different doses.

The salts may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active salts which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the salts to allow for the preparation of highly concentrated solutions.

In addition to the formulations described previously, the salts may also be formulated as a depot preparation. Such long acting formulations may be administered by implantation (for example subcutaneously or intramuscularly) or by intramuscular injection. Thus, for example, the salts may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The salts may also be formulated in rectal compositions such as suppositories or retention enemas, e.g., containing conventional suppository bases such as cocoa butter, polyethylene glycol, or other glycerides.

Salts of the present invention may be administered topically, that is by non-systemic administration. This includes the application of a salt of a compound of any of Formulas I to V externally to the epidermis or the buccal cavity and the instillation of such a salt into the ear, eye and nose, such that the salt does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastimes comprising the active ingredient in a basis such as gelatin and glycerin or sucrose and acacia.

For administration by inhalation the salts according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the salts according to the invention may take the form of a dry powder composition, for example a powder mix of the salt and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The salts of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of salt of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The salts of the subject invention can be administered in various modes, e.g. orally, topically, or by injection. The precise amount administered to a patient will be the responsibility of the attendant physician. The specific dose level for any particular patient will depend upon a variety of factors including the activity of the specific salt employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the salts described herein in combination with another therapeutic agent. By way of example only, if one of the side effects experienced by a patient upon receiving one of the salts herein is hypertension, then it may be appropriate to administer an anti-hypertensive agent in combination with the initial therapeutic agent. Or, by way of example only, the therapeutic effectiveness of one of the salts described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the salts described herein with another therapeutic agent (which also includes a therapeutic regimen) that also has therapeutic benefit. By way of example only, in a treatment for diabetes involving administration of one of the salts described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the salts of the invention with: a) corticosteroids including betamethasone dipropionate (augmented and nonaugmented), betamethasone valerate, clobetasol propionate, diflorasone diacetate, halobetasol propionate, amcinonide, dexosimethasone, fluocinolone acetononide, fluocinonide, halocinonide, clocortalone pivalate, dexosimetasone, and flurandrenalide; b) non-steroidal anti-inflammatory drugs including diclofenac, ketoprofen, and piroxicam; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocaine, and EMLA Cream [Eutectic Mixture of Local Anesthetics (lidocaine 2.5% and prilocaine 2.5%; an emulsion in which the oil phase is a eutectic mixture of lidocaine and prilocaine in a ratio of 1:1 by weight. This eutectic mixture has a melting point below room temperature and therefore both local anesthetics exist as a liquid oil rather then as crystals)]; e) expectorants and combinations thereof with other agents, including guaifenesin and guaifenesin/ketoprofen/cyclobenzaprine; f) antidepressants including tricyclic antidepressants (e.g., amitryptiline, doxepin, desipramine, imipramine, amoxapine, clomipramine, nortriptyline, and protriptyline), selective serotonin/norepinephrine reuptake inhibitors including (e.g, duloxetine and mirtazepine), and selective norepinephrine reuptake inhibitors (e.g., nisoxetine, maprotiline, and reboxetine), selective serotonin reuptake inhibitors (e.g., fluoxetine and fluvoxamine); g) anticonvulsants and combinations thereof, including gabapentin, carbamazepine, felbamate, lamotrigine, topiramate, tiagabine, oxcarbazepine, carbamezipine, zonisamide, mexiletine, gabapentin/clonidine, gabapentin/carbamazepine, and carbamazepine/cyclobenzaprine; h) antihypertensives including clonidine; i) opioids including loperamide, tramadol, morphine, fentanyl, oxycodone, levorphanol, and butorphanol; j) topical counter-irritants including menthol, oil of wintergreen, camphor, eucalyptus oil and turpentine oil; k) topical cannabinoids including selective and non-selective CB1/CB2 ligands; and other agents, such as capsaicin.

In any case, the multiple therapeutic agents (at least one of which is a salt of a compound of any of Formulas I to V, described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

As used in the present specification the following terms have the meanings indicated:

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. More preferred alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an amino group attached to the parent molecular moiety through an alkyl group.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH— group, with R as defined herein.

The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted with one, two, three, four, or five substituents independently selected from the group consisting of alkenyl, alkoxy, alkoxyalkyl, alkyl, cyano, halo, haloalkoxy, haloalkyl, hydroxy, hydroxy-alkyl, nitro, and oxo.

The term “aminoalkyl,” as used herein, alone or in combination, refers to an amino group attached to the parent molecular moiety through an alkyl group. Examples include aminomethyl, aminoethyl and aminobutyl. The term “alkylamino” denotes amino groups which have been substituted with one or two alkyl radicals. Suitable “alkylamino” groups may be mono- or dialkylated, forming groups such as, for example, N-methylamino, N-ethylamino, N,N-dimethylamino, N,N-diethylamino and the like.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(N—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl-O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄═ derived from benzene. Examples include benzothiophene and benzimidazole.

The term “O-carbamyl” as used herein, alone or in combination, refers to a —OC(O)NR, group-with R as defined herein.

The term “N-carbamyl” as used herein, alone or in combination, refers to a ROC(O)NH— group, with R as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.e term “cycloalkyl” embraces radicals having three to ten carbon atoms, such as cyclopropyl cyclobutyl, cyclopentyl, cyclohexyl and cycloheptyl.

The term “ester,” as used herein, alone or in combination, refers to a carbonyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (CFH), difluoromethylene (CF₂), chloromethylene (CHCl) and the like. Examples of such haloalkyl radicals include chloromethyl, 1-bromoethyl, fluoromethyl, difluoromethyl, trifluoromethyl, 1,1,1-trifluoroethyl, perfluorodecyl and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2-NH—OCH3.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; unsaturated condensed heterocyclic groups containing 1 to 2 oxygen atoms and 1 to 3 nitrogen atoms [e.g. benzoxazolyl, benzoxadiazolyl, etc.]; unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.]and isothiazolyl; unsaturated condensed heterocyclic groups containing 1 to 2 sulfur atoms and 1 to 3 nitrogen atoms [e.g., benzothiazolyl, benzothiadiazolyl, etc.] and the like. The term also embraces radicals where heterocyclic radicals are fused with aryl radicals. Examples of such fused bicyclic radicals include benzofuryl, benzothienyl, and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroalkyl” or “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkenyl,” as used herein, alone or in combination, refers to a heterocycle group attached to the parent molecular moiety through an alkenyl group.

The term “heterocycloalkoxy,” as used herein, alone or in combination, refers to a heterocycle group attached to the parent molecular group through an oxygen atom.

The term “heterocycloalkyl,” as used herein, alone or in combination, refers to an alkyl radical as defined above in which at least one hydrogen atom is replaced by a heterocyclo radical as defined above, such as pyrrolidinylmethyl, tetrahydrothienylmethyl, pyridylmethyl and the like.

The term “heterocycloalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., N N.

The term “hydroxy,” as used herein, alone or in combination, refers to —OH.

The term “hydroxyalkyl” as used herein, alone or in combination, refers to a linear or branched alkyl group having one to about ten carbon atoms any one of which may be substituted with one or more hydroxyl radicals. Examples of such radicals include hydroxymethyl, hydroxyethyl, hydroxypropyl, hydroxybutyl and hydroxyhexyl.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′S— group, where R is as defined herein.

The term “mercaptyl” as used herein, alone or in combination, refers to an RS— group, where R is as defined herein.

The term “null” refers to a lone electron pair.

The term “nitro,” as used herein, alone or in combination, refers to —NO₂. The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “oxo” as used herein, alone or in combination, refers to a doubly bonded oxygen.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S and —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NH— group with R as defined herein.

The term “S-sulfonamido” refers to a —S(═O)₂NR₂, group, with R as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thioether,” as used herein, alone or in combination, refers to a thio group bridging two moieties linked at carbon atoms.

The term “thiol,” as used herein, alone or in combination, refers to an SH group.

The term “thiocarbonyl,” as used herein, when alone includes thioformyl —C(S)H and in combination is a —C(S)— group.

The term “N-thiocarbamyl” refers to an ROC(S)NH— group, with R as defined herein.

The term “O-thiocarbamyl” refers to a —OC(S)NR, group with R as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Asymmetric centers exist in the salts of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, or mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the salts of the present invention may exist as geometric isomers. The present invention includes all cis, trans, syn, anti, entgegen (E), and zusammen (Z) isomers as well as the appropriate mixtures thereof. Additionally, salts may exist as tautomers; all tautomeric isomers are provided by this invention. Additionally, the salts of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “optionally substituted” means the anteceding group may be substituted or unsubstituted. When substituted, the substituents of an “optionally substituted” group may include, without limitation, one or more substituents independently selected from the following groups or designated subsets thereof, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH2CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended.

The term R or the term R′, appearing by itself and without a number designation, unless otherwise defined, refers to an optionally substituted moiety selected from the group consisting of alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence.

The term “bond” refers to a covalent linkage between two atoms, or two moieties when the atoms joined by the bond are considered to be part of larger substructure. A bond may be single, double, or triple unless otherwise specified.

The terms, “polymorphs” and “polymorphic forms” and related terms herein refer to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing, for example, one polymorph might be more likely to form solvates or might be difficult to filter and wash free of impurities (i.e., particle shape and size distribution might be different between polymorphs).

Polymorphs of a molecule can be obtained by a number of methods, as known in the art. Such methods include, but are not limited to, melt recrystallization, melt cooling, solvent recrystallization, desolvation, rapid evaporation, rapid cooling, slow cooling, vapor diffusion and sublimation.

Techniques for characterizing polymorphs include, but are not limited to, differential scanning calorimetry (DSC), X-ray powder diffractometry (XRPD), single crystal X-ray diffractometry, vibrational spectroscopy, e.g. IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies.

The term, “solvate,” as used herein, refers to a crystal form of a substance which contains solvent. The term “hydrate” refers to a solvate wherein the solvent is water.

The term, “desolvated solvate,” as used herein, refers to a crystal form of a substance which can only be made by removing the solvent from a solvate.

The term “amorphous form,” as used herein, refers to a noncrystalline form of a substance.

The term “solubility” is generally intended to be synonymous with the term “aqueous solubility,” and refers to the ability, and the degree of the ability, of a compound to dissolve in water or an aqueous solvent or buffer, as might be found under physiological conditions. Aqueous solubility is, in and of itself, a useful quantitative measure, but it has additional utility as a correlate and predictor, with some limitations which will be clear to those of skill in the art, of oral bioavailability. In practice, a soluble compound is generally desirable, and the more soluble, the better. There are notable exceptions; for example, certain compounds intended to be administered as depot injections, if stable over time, may actually benefit from low solubility, as this may assist in slow release from the injection site into the plasma. Solubility is typically reported in mg/mL, but other measures, such as g/g, may be used. Solubilities typically deemed acceptable may range from 1mg/mL into the hundreds or thousands of mg/mL.

The term “prodrug” refers to a compound that is made more active in vivo. The present compounds can also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the combined amount of active ingredients in the combination therapy. This combined amount will achieve the goal of reducing or eliminating the hyperlipidemic condition.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

All references, patents or applications, U.S. or foreign, cited in the application are hereby incorporated by reference as if written herein.

Certain compounds to be combined with suitable counterions to produce the salts which are the subject of the present invention can generally be made according to the following schemes. All IUPAC names used herein were generated using CambridgeSoft's ChemDraw 10.0.

### General Synthetic Methods for Preparing Compounds

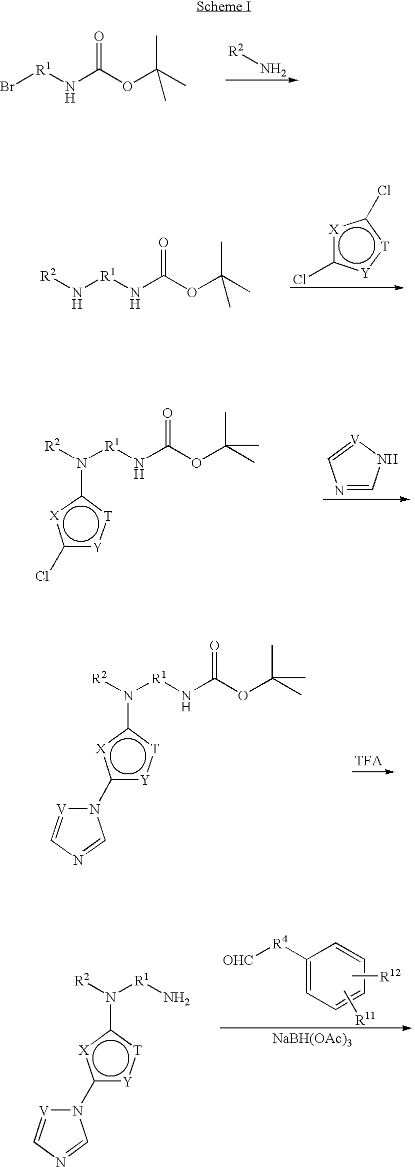

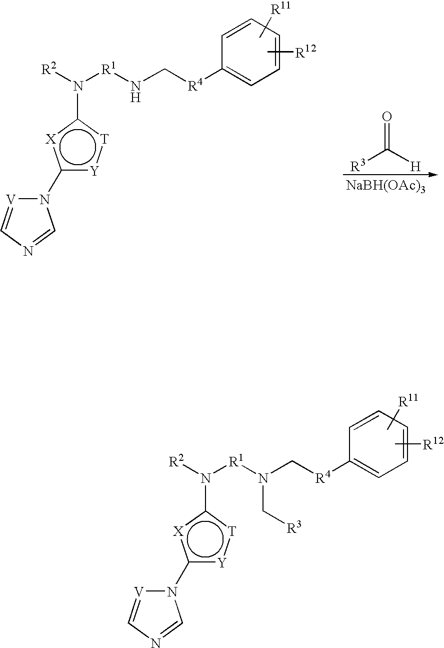

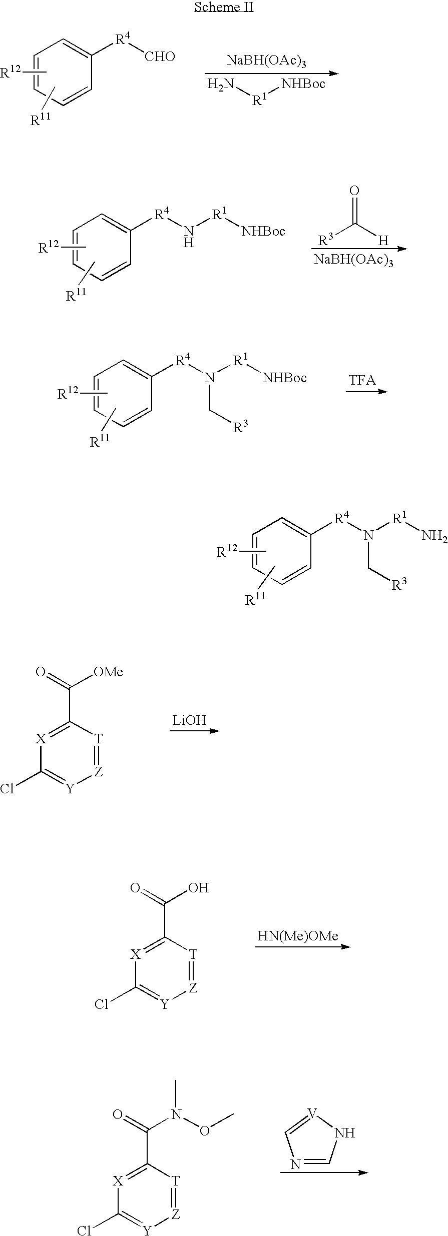

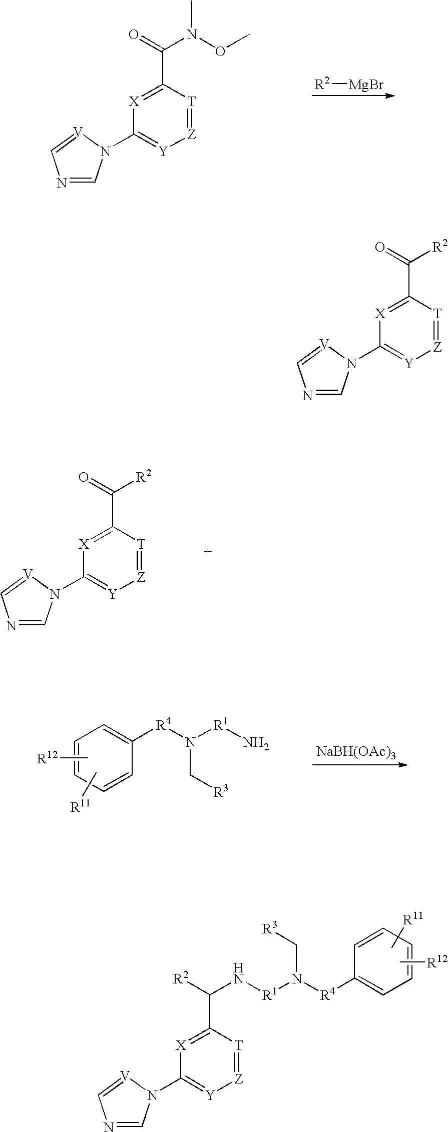

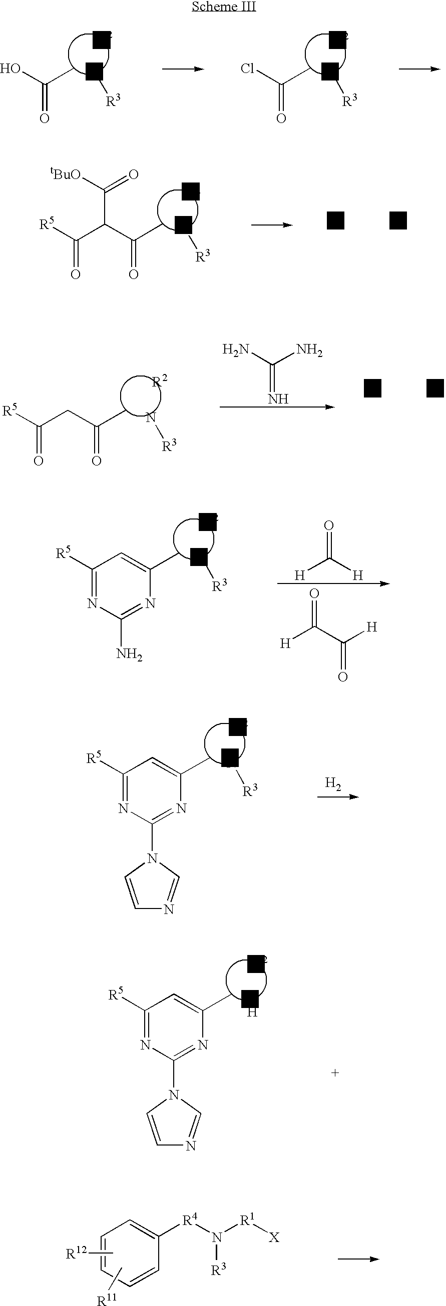

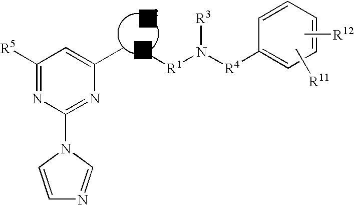

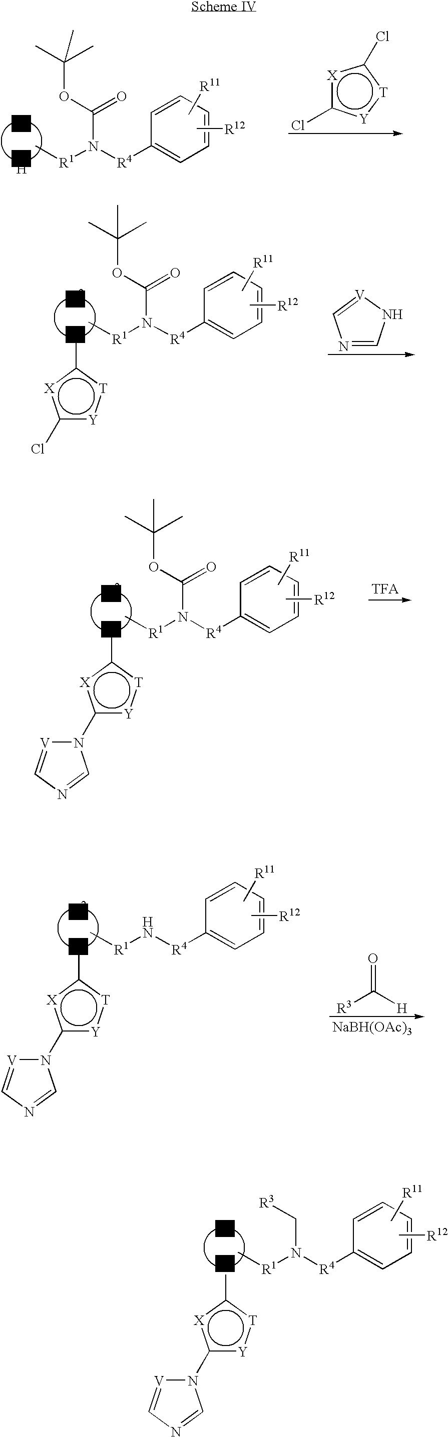

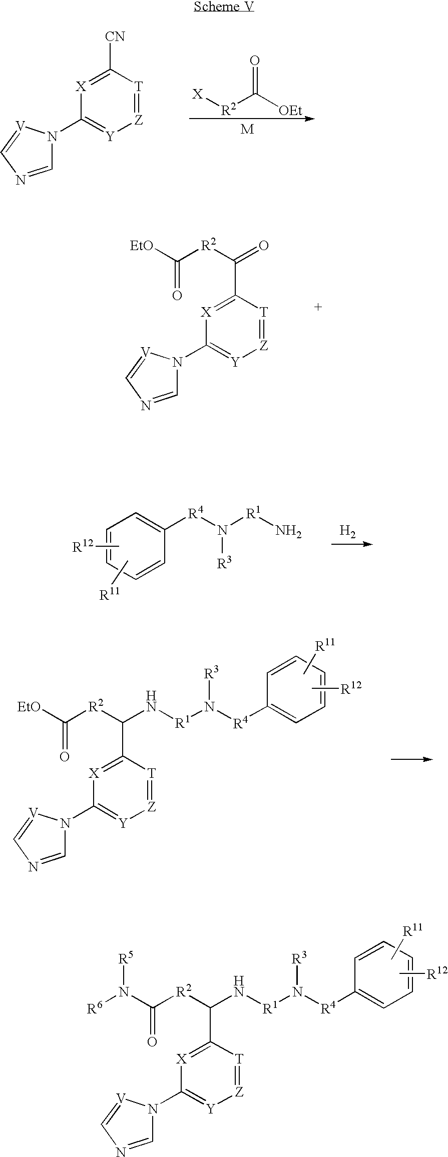

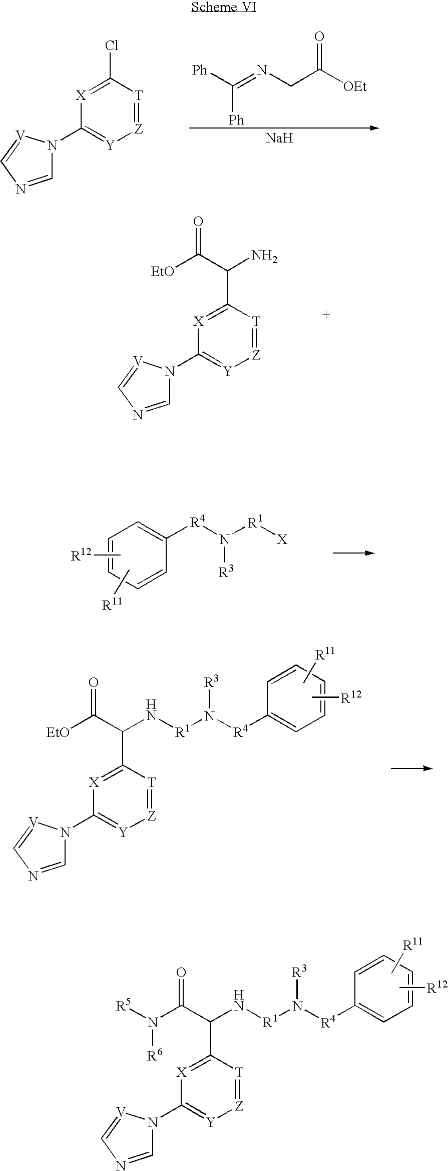

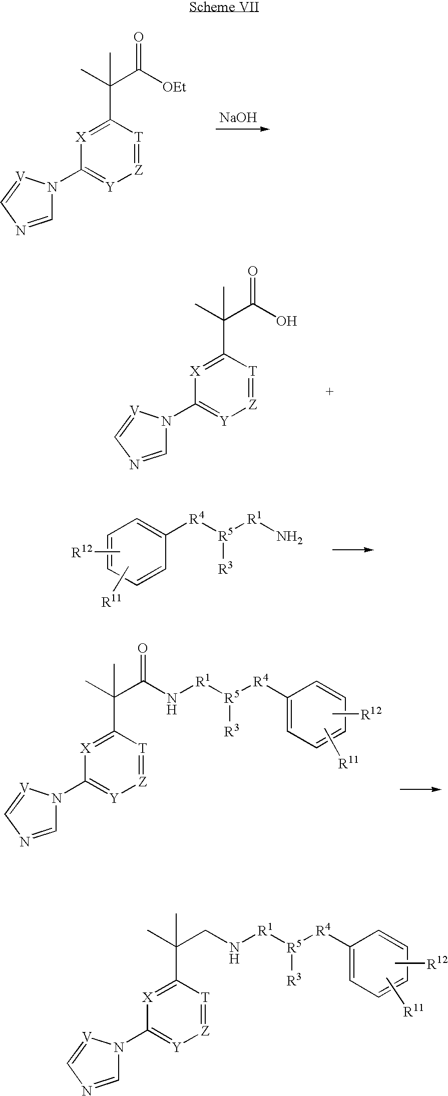

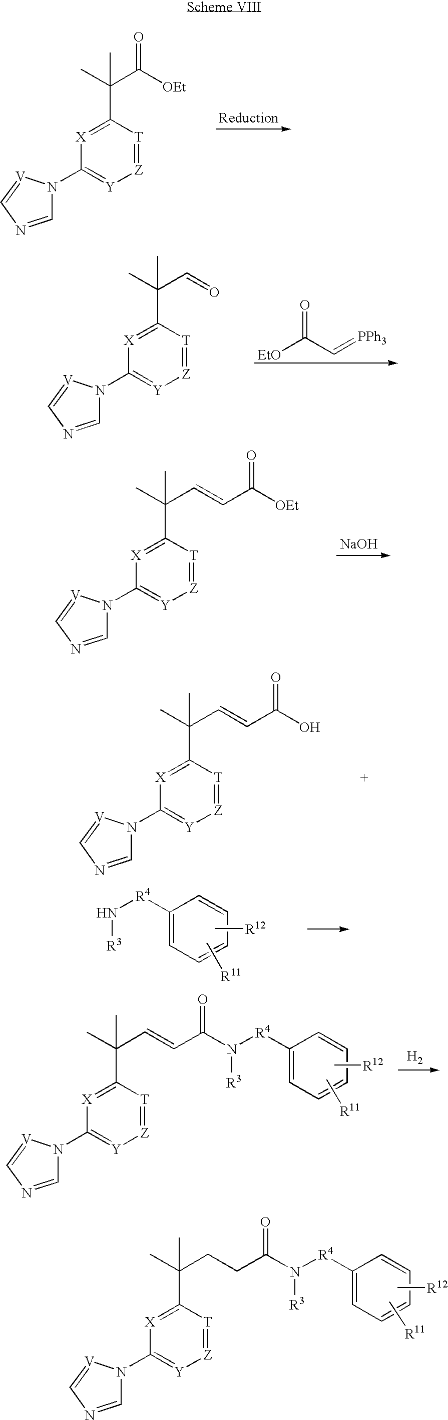

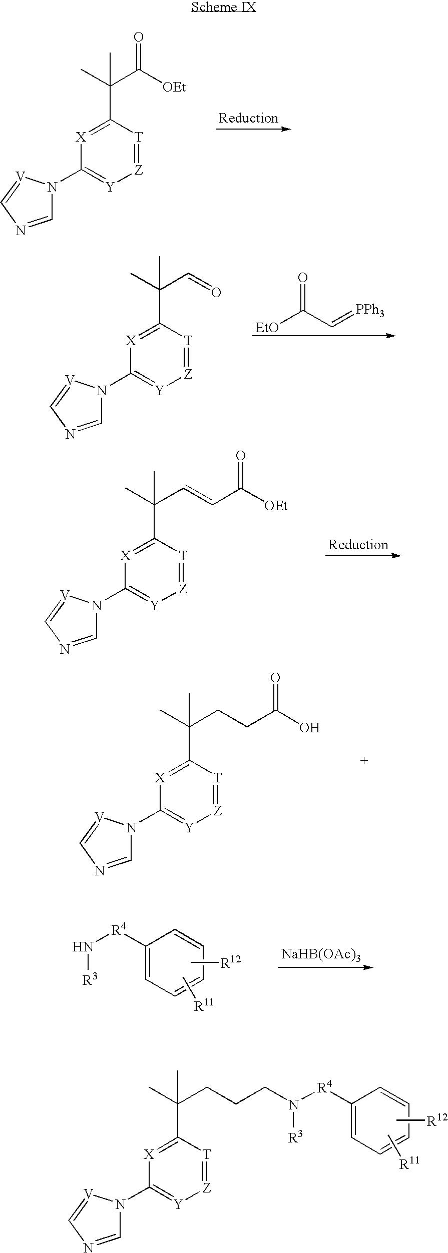

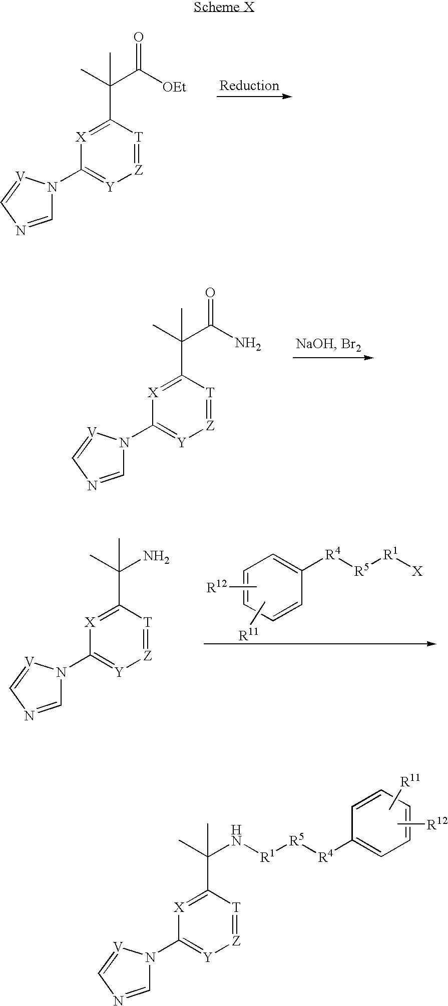

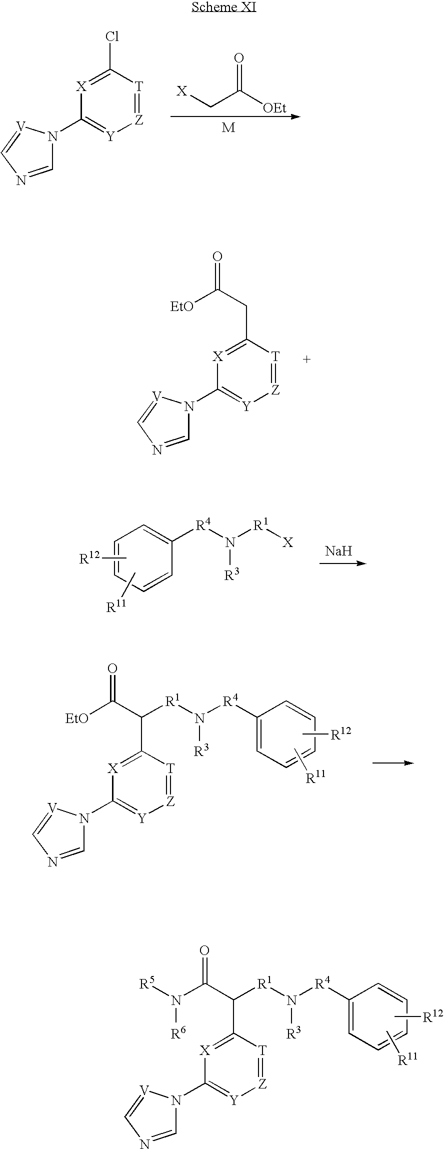

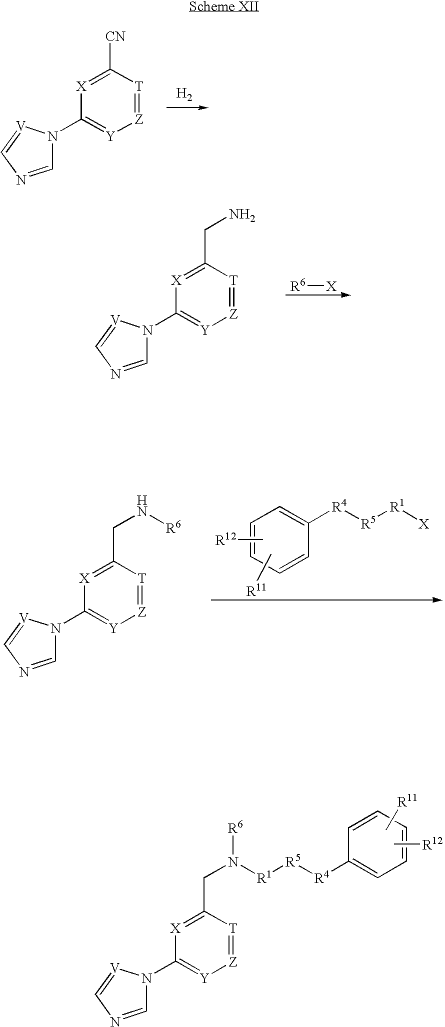

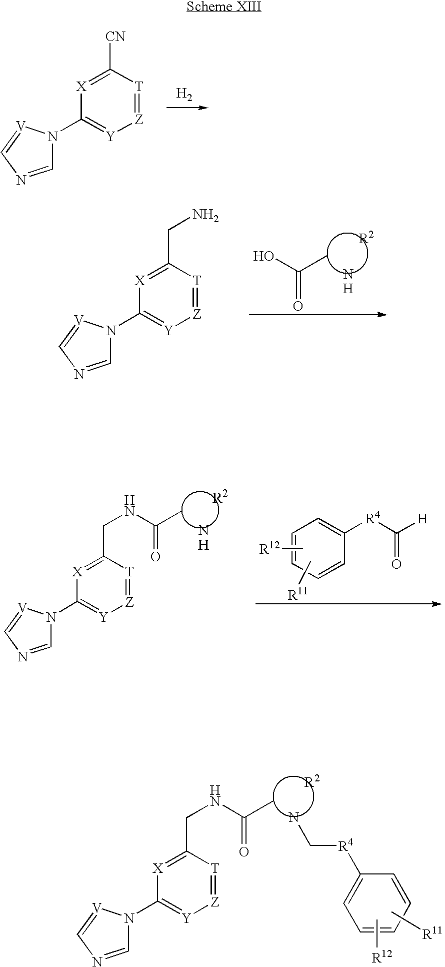

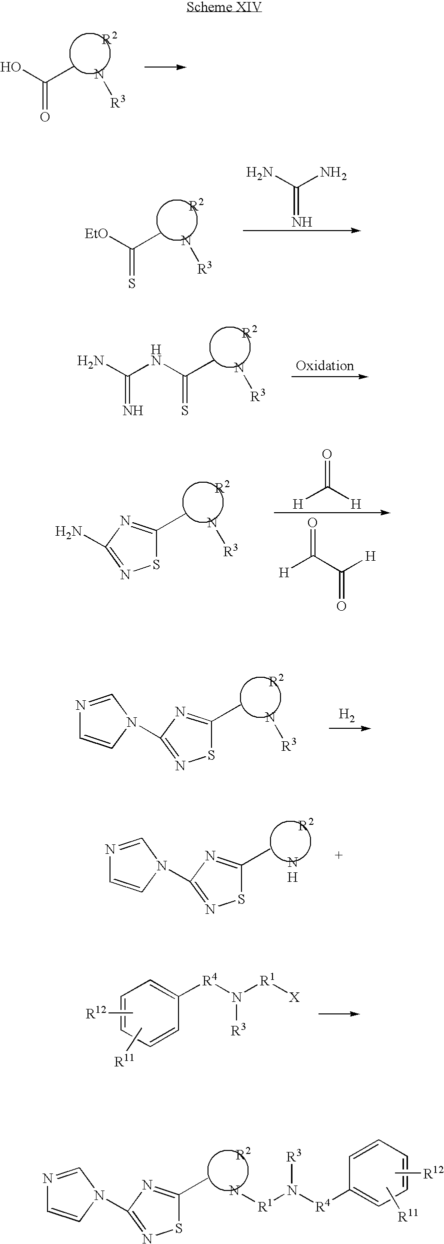

R groups in Schemes I through XIV above are for convenience only, and are intended to represent variability at different positions in the context of a general synthetic scheme, and are not intended to correspond to those defined in Formulas I through V. Likewise, the moiety represented in the Schemes above by a benzyl group substituted with R¹¹ and R¹² should be understood to represent any generic moiety, cyclic or not, heteroatom-containing or not, that one of skill in the art might contemplate as appropriate in such a position. It is consistent for the sake of convenience only in the Schemes above. For a comprehensive description of structural formulas and allowed groups at various positions provided for by the present invention, see the summary of the invention and detailed description of the invention, above.

The invention is further illustrated by the following examples.

### EXAMPLE 1

### Preparation of Compound 1

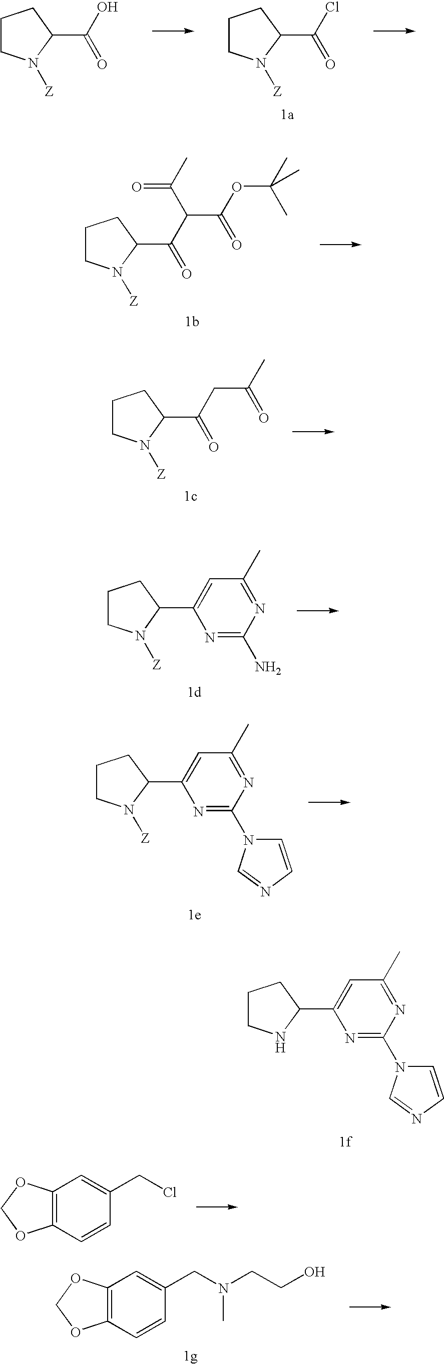 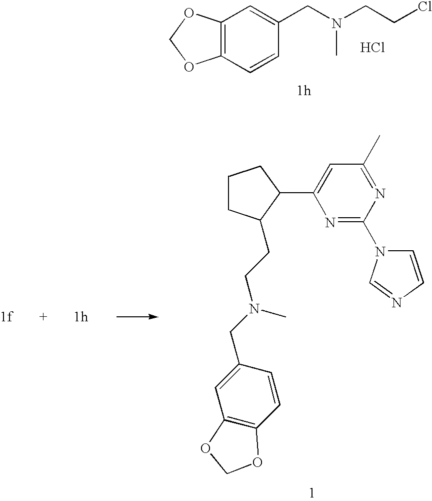 Step 1

### Preparation of compound 1a: 2-Chlorocarbonyl-pyrrolidine-1-carboxylic acid benzyl ester

Oxalyl chloride (707 g, 5.60 mol) was added dropwise (1 h) to a 3° C. solution of N-carbobenzyloxy-D,L-proline (1.00 kg, 4.01 mol), dimethylformamide (0.10 mL) and methylene chloride (4.00 L) under nitrogen. The mixture was warmed to room temperature and stirred for 14 h. The reaction mixture was concentrated to give 1.07 kg (100%) of 2-chlorocarbonyl-pyrrolidine-1-carboxylic acid benzyl ester as an amber oil.

Step 2

### Preparation of compound 1b: 2-(2-tert-Butoxycarbonyl-3-oxo-butyryl)-pyrrolidine-1-carboxylic acid benzyl ester

Methylmagnesium chloride (163 mL of a 3.00 M solution in THF, 489 mmol) was added dropwise to a 4° C. solution of tert-butylacetoacetate (79.0 g, 500 mmol) and THF (500 mL) while maintaining an internal temperature of 4-10° C. The reaction mixture was warmed to 15° C. and 2-chlorocarbonyl-pyrrolidine-1-carboxylic acid benzyl ester (66.0 g, 250 mmol) was added dropwise over 1 h. The mixture was warmed to room temperature and stirred for 12 h. NH₄Cl (300 mL of a saturated aqueous solution) was added and the phases were separated. The organic layer was concentrated under vacuum to give 97.4 g (100%) of 2-(2-tert-butoxycarbonyl-3-oxo-butyryl)-pyrrolidine-1-carboxylic acid benzyl ester as a yellow oil.

Step 3

### Preparation of compound 1c: 2-(3-Oxo-butyryl)-pyrrolidine-1-carboxylic acid benzyl ester

2-(2-tert-Butoxycarbonyl-3-oxo-butyryl)-pyrrolidine-1-carboxylic acid benzyl ester (97.4 g, 250 mmol) was dissolved toluene (400 mL) and was washed with 1N HCl (2×500 mL). p-Toluenesulfonic acid monohydrate (10.0 g, 50.0 mmol) was added to the organic layer and the solution was heated to 80° C. for 4 h under nitrogen. The mixture was cooled to room temperature and water (3×1 L) was added. The phases were separated and the organic layer was concentrated to give 68.7 g (95%) of 2-(3-oxo-butyryl)-pyrrolidine-1-carboxylic acid benzyl ester as an amber oil. [M+H]⁺ 290.03.

Step 4

### Preparation of compound 1d: 2-(2-Amino-6-methyl-pyrimidin-4-yl)-pyrrolidine-1-carboxylic acid benzyl ester

Sodium (5.50 g, 250 mmol) was added portionwise to a stirred solution of anhydrous ethanol (300 mL) under nitrogen at room temperature. A suspension of guanidine hydrochloride (22.8 g, 250 mmol) in ethanol (200 mL) was added and the resulting mixture was stirred for 20 minutes. The precipitate was removed by vacuum filtration and 2-(3-oxo-butyryl)-pyrrolidine-1-carboxylic acid benzyl ester (68.7 g, 237 mmol) was added to the filtrate. The solution was transferred to a flask fitted with a Dean-Stark trap and the reaction mixture was heated to 80° C. The solution was heated at 80° C. under nitrogen for 12 h while removing 200 mL of distillate. The mixture was allowed to cool to room temperature and was gradually cooled to −5° C. The resulting solid was collected by filtration and air dried to give 33.7 g (46%) of 2-(2-amino-6-methyl-pyrimidin-4-yl)-pyrrolidine-1-carboxylic acid benzyl ester as cream colored crystals. [M+H]⁺ 312.88.

Step 5

### Preparation of compound 1e: 2-(2-Imidazol-1-yl-6-methyl-pyrimidin-4-yl)-pyrrolidine-1-carboxylic acid benzyl ester

H₃PO₄ (470 μL) was added to a clear solution of 2-(2-amino-6-methyl-pyrimidin-4-yl)-pyrrolidine-1-carboxylic acid benzyl ester (2.65 g, 8.48 mmol), dioxane (31.2 mL) and water (4.24 mL) at room temperature to give a yellow suspension. Glyoxal (40 wt % in water, 1.23 g, 8.48 mmol), paraformaldehyde (254 mg, 8.48 mmol) and water (8.48 mL) were added and the suspension was heated to 80° C. Saturated NH₄Cl (453 mg, 8.48 mmol in 2.40 mL of H₂O) was added dropwise to the solution at 80° C. prior to heating at 100° C. for 2 h. The mixture was cooled to rt and bought to pH 12 with 4M NaOH then extracted with ethyl acetate. The combined organics were washed with brine and concentrated under vacuum. The product was purified by column chromatography (5:1 ethyl acetate/hexanes) to give 1.98 g (64%) of 2-(2-imidazol-1-yl-6-methyl-pyrimidin-4-yl)-pyrrolidine-1-carboxylic acid benzyl ester as a white solid. [M+H]⁺ 363.78.

Step 6

### Preparation of compound 1f: 2-Imidazol-1-yl-4-methyl-6-pyrrolidin-2-yl-pyrimidine

10% Pd/C (12 mg) was added to a solution of 2-(2-imidazol-1-yl-6-methyl-pyrimidin-4-yl)-pyrrolidine-1-carboxylic acid benzyl ester (112 mg, 0.308 mmol) and ethanol (3 mL) at room temperature. The solution was flushed with nitrogen then stirred under an atmosphere of hydrogen for 4 h. The reaction mixture was filtered through celite and concentrated under vacuum. The product was purified by column chromatography (DCM to 20% MeOH/DCM) to give 63 mg (89%) of 2-imidazol-1-yl-4-methyl-6-pyrrolidin-2-yl-pyrimidine. [M+H]⁺ 230.16; ¹H-NMR (400 MHz, CD₃OD) δ 8.74 (s, 1H), 8.05 (s, 1H), 7.31 (s, 1H), 7.14 (s, 1H), 4.95 (s, 2H), 4.25 (t, 1H), 3.25 (m, 1H), 3.05 (m, 1H), 2.59 (s, 3H), 2.35 (m, 1H), 1.90 (m, 2H); ¹³C-NMR (100 MHz, CD₃OD) δ 173.4, 170.4, 153.9, 136.0, 128.9, 116.8, 116.0, 62.1, 46.5, 32.7, 25.3, 22.7.

Step 7

### Preparation of compound 1g: 2-(Benzo[1,3]dioxol-5-ylmethyl-methyl-amino)-ethanol

2-(Methylamino)ethanol (22.0 g, 290 mmol) was added to a stirred solution of 3,4-methylenedioxybenzyl chloride (25.0 g, 147 mmol) in DCM (45 mL) at −78° C. under nitrogen. The solution was stirred for 15 minutes at −78° C. then warmed to room temperature and stirred for 16 h. 1.2 M NaOH (100 mL) was added and the phases were separated. The organic layer was washed water (2×150 mL) and concentrated under vacuum to give 25.3 g (83%) of 2-(benzo[1,3]dioxol-5-ylmethyl-methyl-amino)-ethanol as a clear oil.

Step 8

### Preparation of compound 1h: Benzo[1,3]dioxol-5-ylmethyl-(2-chloro-ethyl)-methyl-amine hydrochloride salt

Thionyl chloride (60 mL) was added dropwise over 30 minutes to a 0° C. solution of 2-(benzo[1,3]dioxol-5-ylmethyl-methyl-amino)-ethanol (22.2 g, 110 mmol) in DCM (250 mL) under nitrogen. The solution was warmed to room temperature and stirred for 16 h. The suspension was concentrated under vacuum and brine (150 mL) and ethyl acetate (200 mL) were added. The precipitate was collected by vacuum filtration and washed with ethyl acetate (100 mL). The solid was dried overnight under vacuum to give 26.5 g (91%) of benzo[1,3]dioxol-5-ylmethyl-(2-chloro-ethyl)-methyl-amine hydrochloride as a white powder.

Step 9

### Preparation of compound 1: Benzo[1,3]dioxol-5-ylmethyl-{2-[2-(2-imidazol-1-yl-6-methyl-pyrimidin-4-yl)-pyrrolidin-1-yl]-ethyl}-amine

A solution of 2-imidazol-1-yl-4-methyl-6-pyrrolidin-2-yl-pyrimidine (2.1 g, 9.2 mmol) in DMF (15 mL) was added to a stirred mixture of benzo[1,3]dioxol-5-ylmethyl-(2-chloro-ethyl)-methyl-amine hydrochloride salt (2.2 g, 8.1 mmol), DMF (10 mL) and diisopropylethylamine (2.5 mL) at room temperature under nitrogen. Potassium iodide (340 mg, 2.0 mmol) was added and the mixture was heated to 80° C. for 3 h. The solution was cooled to room temperature and 1N dibasic potassium phosphate solution (200 mL) was added. The solution was extracted with ethyl acetate and the phases were separated. The organic layer was concentrated and the product was purified by column chromatography (DCM to 4:1 DCM/MeOH) to give 2.0 g (52%) of benzo[1,3]dioxol-5-ylmethyl-{2-[2-(2-imidazol-1-yl-6-methyl-pyrimidin-4-yl)-pyrrolidin-1-yl]-ethyl}-amine as a red oil. [M+H]⁺ 421.30; ¹H-NMR (400 MHz, CDCl₃) δ 8.60 (s, 1H), 7.89 (s, 1H), 7.30 (s, 1H), 7.10 (s, 1H), 6.78 (s, 1H), 6.67 (m, 2H), 5.88 (s, 2H), 3.52 (t, 1H), 3.6 (m, 3H), 2.77 (m, 1H), 2.2-2.6 (m, 8H), 2.35 (s, 3H), 1.62-1.95 (m, 3H); ¹³C-NMR (100 MHz, CDCl₃) δ 175.7, 169.6, 154.0, 147.6, 146.5, 136.2, 132.8, 130.1, 121.9, 116.6, 115.0, 109.2, 107.8, 100.8, 69.8, 62.3, 56.0, 54.3, 53.1, 42.5, 33.2, 24.2, 23.4.

### EXAMPLE 2

Preparation of compound 1 enantiomer 1: Benzo[1,3]dioxol-5-ylmethyl-{2-[2-(2-imidazol-1-yl-6-methyl-pyrimidin-4-yl)-pyrrolidin-1-yl]-ethyl}-amine was prepared following the procedures described in preparation of Example 1. A single enantiomer of Example 1 was obtained by chiral HPLC (chiralpak ADRH, 4.6×150 mm, 10 mM NH₄OAc/EtOH 4:6 (v/v), flow rate 0.5 mL/min) separation. Analytical data are identical to Example 1.

### EXAMPLE 3

Preparation of compound 1 enantiomer 2: Benzo[1,3]dioxol-5-ylmethyl-{2-[2-(2-imidazol-1-yl-6-methyl-pyrimidin-4-yl)-pyrrolidin-1-yl]-ethyl}-amine was prepared following the procedures described in preparation of Example 1. A single enantiomer of Example 1 was obtained by chiral HPLC (chiralpak ADRH, 4.6×150 mm, 10 mM NH₄OAc/EtOH 4:6 (v/v), flow rate 0.5 mL/min) separation. Analytical data are identical to Example 1.

### EXAMPLE 4

### Preparation of Compound 2

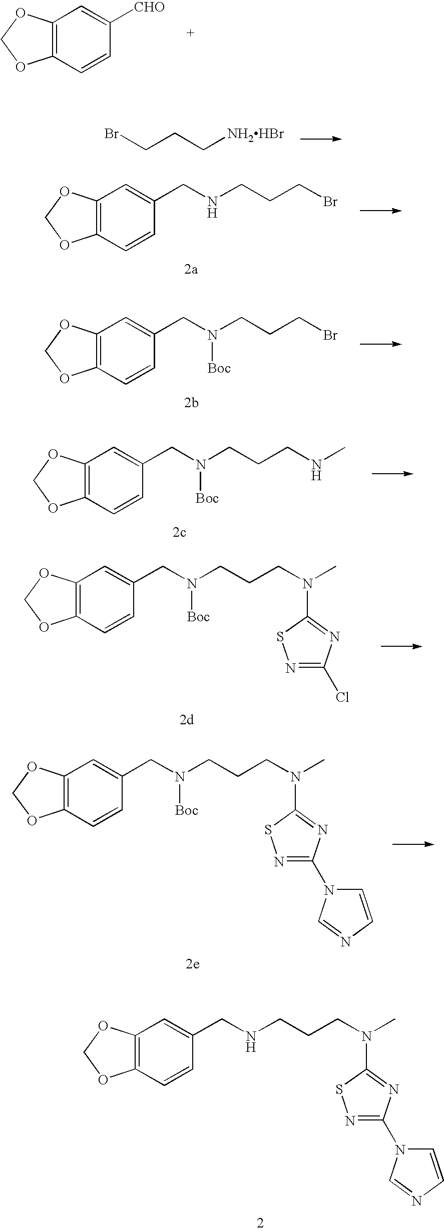 Step 1

### Preparation of compound 2a: Benzo[1,3]dioxol-5-ylmethyl-(3-bromo-propyl)-amine

Triethylamine (1.30 L, 9.30 mol) was added to a suspension of 3-bromopropan-1-amine hydrobromide (2.00 kg, 9.10 mol) in CH₂Cl₂ (16.0 L) at 22° C. under nitrogen. The solution was stirred for 15 minutes prior to the addition of piperonal (1.30 kg, 8.70 mol). The mixture was heated to 40° C. for 2.5 h and cooled to room temperature. Water (9.00 L) was added to the suspension and the mixture was stirred for 20 minutes. The layers were separated and organic layer was concentrated under vacuum to a yellow oil. Isopropanol (16.0 L) and acetic acid (1.50 L) were added to the oil. The solution was cooled to 15° C. under nitrogen and sodium triacetoxyborohydride (2.20 kg, 10.4 mol) was added in 50 g portions over 1 h. The mixture was stirred at room temperature for 14 h prior to cooling to 15° C. Water (6 L) was added while maintaining an internal temperature below 26° C. The pH was adjusted to 7-8 with the sat. aqueous K₂CO₃ followed by the addition of brine (10.0 L). The precipitate was collected by vacuum filtration and washed with water (10.0 L). The solid was dried overnight under vacuum to afford 1.24 kg (53%) of benzo[1,3]dioxol-5-ylmethyl-(3-bromo-propyl)-amine as a white solid. [M+H]⁺ 271.90, 273.94; ¹H-NMR (400 MHz, DMSO) δ 7.25 (s, 1H), 7.04 (d, 1H), 6.96 (d, 1H), 6.05 (s, 2H), 4.04 (s, 2H), 3.61 (t, 2H), 2.94 (t, 2H), 2.24 (t, 2H); ¹³C-NMR (100 MHz, DMSO) δ 148.1, 147.7, 126.1, 124.6, 110.8, 108.7, 101.8, 50.1, 45.2, 31.9, 29.1

Step 2

### Preparation of compound 2b: Benzo[1,3]dioxol-5-ylmethyl-(3-bromo-propyl)-carbamic acid tert-butyl ester

Triethylamine (1.24 L, 8.90 mol) was added over 45 minutes to a mixture of benzo[1,3]dioxol-5-ylmethyl-(3-bromo-propyl)-amine (2.20 kg, 8.10 mol) and di-tert-butyl dicarbonate (1.94 kg, 8.90 mol) in MeOH (20.0 L) at 20-24° C. under nitrogen. The solution was stirred for 1 h at room temperature. The mixture was concentrated under vacuum (70-15 torr) at 32° C. prior to the addition of ethyl acetate (5.00 L) and water (3.00 L). The layers were separated and the aqueous back extracted with ethyl acetate (1.00 L). The combined organic layers were concentrated under vacuum (70-5 torr) at 32° C. to give 2.93 kg (97%) of benzo[1,3]dioxol-5-ylmethyl-(3-bromo-propyl)-carbamic acid tert-butyl ester as an amber oil.

Step 3

### Preparation of compound 2c: Benzo[1,3]dioxol-5-ylmethyl-(3-methylamino-propyl)-carbamic acid tert-butyl ester

Methylamine (33 wt. % in EtOH, 30.0 L, 240 mol) was added over 3 h to a solution of benzo[1,3]dioxol-5-ylmethyl-(3-bromo-propyl)-carbamic acid tert-butyl ester (2.93 kg, 7.90 mol) in EtOH (4.00 L) while maintaining an internal temperature of 14-17° C. The reaction mixture was warmed to room temperature and stirried for 14 h. The solution was concentrated under vacuum (70-15 torr) at 32° C. then partitioned between ethyl acetate (5.00 L) and water (3.00 L). The phases were separated and the aqueous layer back extracted with ethyl acetate (2.00 L). The combined organic layers were concentrated under vacuum (70-5 torr) at 32° C. to give 2.59 kg (100%) of benzo[1,3]dioxol-5-methyl-(3-methylamino-propyl)-carbamic acid tert-butyl ester as a clear oil. [M+H]⁺ 323.70.

Step 4

### Preparation of compound 2d: Benzo[1,3]dioxol-5-ylmethyl-{3-[(3-chloro-[1,2,4]thiadiazol-5-yl)-methyl-amino]-propyl}-carbamic acid tert-butyl ester

A solution of benzo[1,3]dioxol-5-ylmethyl-(3-methylamino-propyl)-carbamic acid tert-butyl ester (2.59 kg, 7.90 mol) in CH₂Cl₂ (20.0 L) was cooled to 7.5° C. under nitrogen. Triethylamine (2.20 L, 15.8 mol) was added and the solution was cooled to 0.5° C. 3,5-Dichloro-1,2,4-thiadiazole (1.22 kg, 7.90 mol) was added over 2 h while maintaining an internal temperature of 0-2° C. The reaction mixture was warmed room temperature and stirred for 15 h. Water (9.00 L) was added and the organic layer was separated. The solution was concentrated under vacuum (220-10 torr) at 32° C. to give 3.26 kg (94%) of benzo[1,3]dioxol-5-ylmethyl-{3-[(3-chloro-[1,2,4]thiadiazol-5-yl)-methyl-amino]-propyl}-carbamic acid tert-butyl ester as an amber oil. [M+H]⁺ 441.37; ¹H-NMR (400 MHz, CD₃OD) δ 6.77 (m, 3H), 5.96 (s, 2H), 4.35 (s, 2H), 3.4-3.0 (m, 6H), 1.84 (br s, 3H), 1.50 (s, 9H).

Step 5

### Preparation of compound 2e: Benzo[1,3]dioxol-5-ylmethyl-{3-[(3-imidazol-1-yl-[1,2,4]thiadiazol-5-yl)-methyl-amino]-propyl}-carbamic acid tert-butyl ester

Sodium imidazole (2.10 kg, 23.1 mol) was added to a solution of benzo[1,3]dioxol-5-ylmethyl-{3-[(3-chloro-[1,2,4]thiadiazol-5-yl)-methyl-amino]-propyl}-carbamic acid tert-butyl ester (3.00 kg, 6.80 mol) in DMSO (8.00 L) at 22° C. under nitrogen. The solution was heated at 74° C. for 13 h then cooled to room temperature and stirred for 7 h. Citric acid (10 L of a 5% aqueous solution) was added over 8 hours and the solution was extracted with ethyl acetate (10.0 L). The layers were separated and the organic layer was concentrated to give 3.18 kg (99%) of benzo[1,3]dioxol-5-ylmethyl-{3-[(3-imidazol-1-yl-[1,2,4]thiadiazol-5-yl)-methyl-amino]-propyl}-carbamic acid tert-butyl ester as a green oil. [M+H]⁺ 473.06; ¹H-NMR (400 MHz, CD₃OD) δ 8.32 (s, 1H), 7.68 (s, 1H), 7.12 (s, 1H), 6.62-6.80 (m, 3H), 5.96 (s, 2H), 4.38 (s, 2H), 3.0-3.6 (m, 6H), 1.88 (br s, 3H), 1.52 (s, 9H).

Step 6

### Preparation of compound 2: N′-Benzo[1,3]dioxol-5-ylmethyl-N-(3-imidazol-1-yl-[1,2,4]thiadiazol-5-yl)-N-methyl-propane-1,3-diamine

A solution of benzo[1,3]dioxol-5-ylmethyl-{3-[(3-imidazol-1-yl-[1,2,4]thiadiazol-5-propyl}-carbamic acid tert-butyl ester (10.6 g, 22.4 mmol) in a mixture of TFA/DCM (70 mL of a 1:1 mixture) was stirred at room temperature for 30 min. The solution was concentrated under vacuum and K₂CO₃ (50 mL of a saturated aqueous solution) was added. The mixture was extracted with ethyl acetate (2×200 mL) and the combined organics were concentrated under vacuum to give 8.30 g (99%) of N′-benzo[1,3]dioxol-5-ylmethyl-N-(3-imidazol-1-yl-[1,2,4]thiadiazol-5-yl)-N-methyl-propane-1,3-diamine as a colorless oil. [M+H]⁺ 373.26; ¹H-NMR (400 MHz, CD₃OD) δ 8.28 (s, 1H), 7.63 (s, 1H), 7.07 (s, 1H), 6.79 (s, 1H), 6.72 (s, 2H), 5.92 (s, 2H), 3.67 (s, 3 H), 3.60 (br s, 1H), 3.10 (br s, 2H), 2.66 (t, 2H), 2.0 (br s, 2H), 1.87 (q, 2 H).

### EXAMPLE 5

### Preparation of Hydrochloride Salt of Compound 2

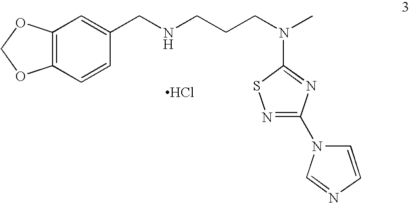

### Preparation of compound 3: N′-Benzo[1,3]dioxol-5-ylmethyl-N-(3-imidazol-1-yl-[1,2,4]thiadiazol-5-yl)-N-methyl-propane-1,3-diamine hydrochloride salt

A suspension of 1 (11.4 g, 30.6 mmol) in EtOH (60 mL) was heated to 55° C. for 15 minutes to afford a clear solution. Concentrated HCl (2.63 mL, 31.5 mmol) was added causing immediate precipitation. The suspension was stirred for an additional 15 minutes at 55° C. then n-heptane (110 mL) was added and the mixture was cooled to room temperature. The precipitate was collected by vacuum filtration and washed with n-heptanes (30 mL) to afford 11.19 g (90%) of 2 as a white solid. [M+H]⁺ 373.13; ¹H-NMR (400 MHz, DMSO) δ 9.59 (s, 2H), 8.14 (s, 1H), 7.67 (s, 1H), 7.20 (s, 1H), 6.98 (d, 1H), 6.87 (d, 1H), 6.01 (s, 2H), 4.00 (t, 2 H), 3.82-3.68 (br s, 2H), 3.20-3.00 (br s, 3H), 2.86 (m, 2H), 2.09 (quint, 2H);

Elemental found (calc) C, 49.70 (49.93); H, 5.17 (5.18); N, 20.36 (20.55); S, 7.78 (7.84); Cl, 8.89 (8.67).

### EXAMPLE 6

### Preparation of Acetate Salt of Compound 2

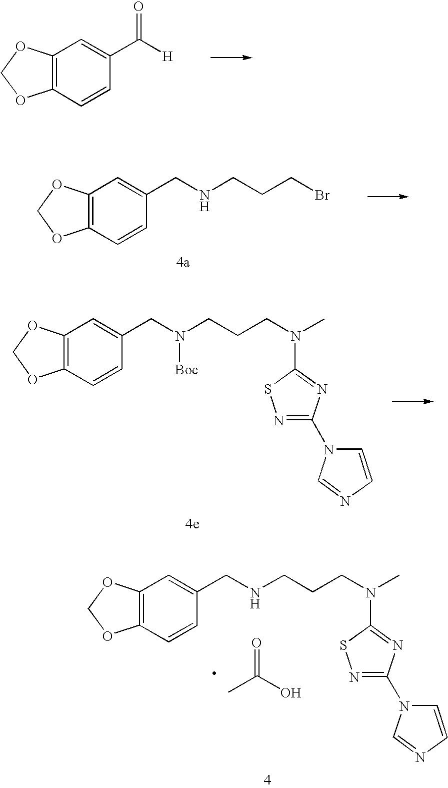 Step 1

### Preparation of compound 4a: Benzo[1,3]dioxol-5-ylmethyl-(3-bromo-propyl)-amine

Isopropanol (24.0 L) was added to a nitrogen purged reactor charged with piperonal (3.018 kg, 20.12 mol) and 3-bromopropan-1-amine hydrobromide (4.3995 kg, 20.10 mol). The resulting suspension was stirred until complete dissolution was observed (30 minutes) prior to the addition of triethylamine (2.0357 kg, 20.12 mol) via a feeding vessel. The feeding vessel was rinsed with isopropanol (0.800 L) and added to the reaction mixture. The mixture was stirred at 20° C. for 43 minutes and the resulting suspension was filtered. The vessel and filtered cake were washed with isopropanol (2×7.500 L) and combined with the mother liquor. The solution was transferred to a reactor and cooled to 5° C. prior to the addition of acetic acid (3.622 kg, 60.34 mol). NaHB(OAc)₃ (5.3670 kg, 25.32 mol) was added in ten portions over 51 minutes via a Müller barrel while maintaining an internal temperature of 5.2-9.6° C. The mixture was warmed to 22.0° C., stirred for 35 minutes then cooled to 14.6° C. Water (75.0 L) was slowly added to the mixture while maintaining an internal temperature of 14.6-21.1° C. The pH of the solution was adjusted to 7-8 with the addition of K₂CO₃ (18.0 L of a 19.4% aqueous solution) at an internal temperature of 21.1° C. Sodium chloride (37.0 L of a 23.1% aqueous solution) was added causing mass precipitation. The mixture was stirred for 30 minutes before filtration of the precipitate. The vessel and the filter cake were rinsed with water (2×30.0 L). The filter cake was dried under nitrogen and transferred into a tarred flask. The solid was dried for 44.25 hours, using a rotary evaporator, at a bath temperature of 40° C. and a pressure of 8 mbar, to give 3.778 kg (69%) of benzo[1,3]dioxol-5-ylmethyl-(3-bromo-propyl)-amine as an off-white solid. [M+H]⁺ 271.90, 273.94; ¹H-NMR (400 MHz, DMSO) δ 7.25 (s, 1H), 7.04 (d, 1H), 6.96 (d, 1H), 6.05 (s, 2H), 4.04 (s, 2H), 3.61 (t, 2H), 2.94 (t, 2H), 2.24 (t, 2H); ¹³C-NMR (100 MHz, DMSO) δ 148.1, 147.7, 126.1, 124.6, 110.8, 108.7, 101.8, 50.1, 45.2, 31.9, 29.1

Step 2

### Preparation of compound 4e: Benzo[1,3]dioxol-5-ylmethyl-{3-[(3-imidazol-1-yl-[1,2,4]thiadiazol-5-yl)-methyl-amino]-propyl}-carbamic acid tert-butyl ester

1a (4.05 kg, 14.9 mol) and Boc₂O (3.26 kg, 14.9 mol) were added to a nitrogen purged 160 L reactor followed by the addition of methanol (45.0 L) via a feeding vessel. A solution of triethylamine (1.51 kg, 14.9 mol) and methanol (11.0 L) was added over a period of 21 min to the reaction mixture, and the resulting solution was maintained at an internal temperature of 20-21° C. for 45 min. The reaction mixture was transferred to the feeding vessel and the reactor was washed with methanol (11.0 L) and combined with the reaction mixture. The reactor, equipped with a 6N sulfuric acid filled scrubber, was charged with a solution of methylamine in ethanol (8N, 55.5 L, 444 mol) and the reaction mixture was slowly added from the feeding vessel over 2.1 h while maintaining an internal temperature of 20-21° C. The solution was maintained at an internal temperature of 20° C. for 37.5 h before removal of 45.0 L of solvent by vacuum distillation, using an external vacuum pump connected to the scrubber, at a pressure ranging from 271 to 45 mbar and a jacket temperature of 49° C., to afford an oil. DCM (16.0 L) and an aqueous solution of Na₂CO₃ (9.5%, 32.4 L) were added to the oil and stirred at 19-21° C. for 13 minutes. The separated aqueous layer was back extracted with DCM (16.0 L) and the combined organic layers were washed with water (16.0 L). The separated organics were concentrated through azeotropic distillation, at an internal temperature of 22-23° C. and a pressure of 503-501 mbar, to yield a pale brown solution. The solution and TEA (4.74 kg, 46.8 mol) were charged into a nitrogen purged 160 L reactor. A solution of 3,5-dichloro-1,2,4-thiadiazole (2.49 kg, 16.1 mol) in DCM (20.0 L) was added to the reaction mixture from the feeding vessel, over 48 min, while maintaining an internal temperature of 18-22° C. The reaction mixture was maintained at 18-20° C. for 16.4 hours followed by addition of water (40 L) and the resulting mixture was stirred at an internal temperature of 20° C. for 7 min. To the separated organic layer was added an aqueous solution of NaCl (half saturated, 20 L). The resulting mixture was stirred for 6 min at an internal temperature of 20° C. before transferring the organic layer into the reaction vessel and removing 55 L of solvent by distillation at an internal temperature of 19-28° C. and a pressure of 500-300 mbar. Residual DCM was removed by iterative distillation with TBME (3×41 L) at an internal temperature of 14-27° C. and a pressure of 244-75 mbar. DMSO (35 L) was added and the vacuum was released, yielding a solution. Sodium imidazole (4.22 kg, 46.9 mol) was added and the resulting mixture was heated to an internal temperature of 80° C. over 2.13 hours and maintained at 80° C. for an additional 9.85 h. The reaction was then cooled to 20° C. followed by addition of water (35 L) over 1 h at an internal temperature of 20-23° C. ⁱPrOAc (35 L) was added and the mixture was stirred for 6 minutes. The separated aqueous layer was extracted with ⁱPrOAc (17 L) and the combined organic layers were washed sequentially with brine (34 L), citric acid (34 L of a 5% aqueous solution) and brine (18 L). The organic layer was concentrated to an oil by distillation at an internal temperature of 19-27° C. and a pressure of 195-64 mbar. The oil was dried for 71.5 hours at an external temperature of 20-40° C. and a pressure of 53-8 mbar prior to manual removal of paraffin oil (0.341 kg) to give 6.55 kg (93%) of benzo[1,3]dioxol-5-ylmethyl-{3-[(3-imidazol-1-yl-[1,2,4]thiadiazol-5-yl)-methyl-amino]-propyl}-carbamic acid tert-butyl ester as a pale brown oil.

[M+H]⁺ 473.06; ¹H-NMR (400 MHz, CD₃OD) δ 8.32 (s, 1H), 7.68 (s, 1H), 7.12 (s, 1H), 6.62-6.80 (m, 3H), 5.96 (s, 2H), 4.38 (s, 2H), 3.0-3.6 (m, 6H), 1.88 (br s, 3H), 1.52 (s, 9H).

Step 3

### Preparation of compound 4: N′-Benzo[1,3]dioxol-5-ylmethyl-N-(3-imidazol-1-yl-[1,2,4]thiadiazol-5-yl)-N-methyl-propane-1,3-diamine acetate salt

1e (6.69 kg, 14.2 mol) was dissolved in isopropanol (1.34 L) and TBME (5.5 L). The resulting solution was added to a nitrogen purged 160 L reactor, equipped with a scrubber filled with water (40.0 L), and the feeding vessel was rinsed with TBME (48.0 L). The rinsing solvent was added to the reactor and the solution was heated to 35° C. over 22 min. A solution of HCl (8.07 kg, 221 mol) in water (2.0 L) was added to the reaction mixture over 32 min at an internal temperature of 34-37° C. The reaction mixture was maintained for 1 h at 34-37° C. with subsequent cooling to 19 ° C. The organic layer was discarded and the aqueous layer was treated with methanol (8.0 L) and TBME (72.0 L). An aqueous solution of K₂CO₃ (25%, 53.5 L) was added over 20 min at an internal temperature of 20-23° C. and the mixture was stirred for 1 h at 20-23° C. The layers were separated and the aqueous layer was back extracted with a mixture of methanol (2.8 L) and TBME (24.0 L). The combined organic layers were added to solid Na₂CO₃ (0.838 kg, 9.97 mol) and stirred for 12 minutes. The resulting suspension was filtered and the filter cake was washed with TBME (6.0 L). The filtrate was transferred to the reactor and 99.0 L of solvent was removed by distillation at an internal temperature of 20-36° C. and a pressure of 304-203 mbar. Isopropanol (27.0 L) was added and 27.5 L of solvent was removed by distillation at an internal temperature of 32-40° C. and a pressure of 94-44 mbar. Additional isopropanol (25.5 L) was added and the solution was filtered twice through an inline filter and heated to 55° C. Sequential addition, through inline filtration, of acetic acid (0.871 kg, 14.5 mol) and isopropanol (0.350 L) afforded a suspension that was stirred for 30 min at an internal temperature of 55° C. prior to addition of heptanes (51.0 L), through an inline filter, at an internal temperature of 51-56° C. The reaction mixture was slowly cooled to 20° C. over 4.5 h and maintained at an internal temperature of 20° C. for 9.67 hours. The resulting suspension was filtered, the reactor and filter cake were rinsed with inline filtered heptanes (2×16.0 L) and the filter cake was dried with a stream of nitrogen for 3 h. The solid was dried at an external temperature of 35-45° C. and a pressure of 53-8 mbar for 20 hours, affording 4.38 kg (72%) of 4 as a white to off-white solid. [M+H]⁺ 373.40; ¹H-NMR (400 MHz, DMSO) δ 8.28 (s, 1H), 7.70 (s, 1H), 7.06 (s, 1H), 6.90 (d, 1H), 6.80 (d, 1H), 6.76 (d, 1H), 5.95 (s, 2H), 3.65 (s, 2H), 3.70-3.54 (br s, 1H), 3.20-3.04 (br s, 4H), 2.56 (t, 2H), 2.47 (m, 2H), 1.89 (s, 3H), 1.81 (quint, 2H); Elemental found (calc) C, 52.80 (52.76); H, 5.58 (5.59); N, 19.40 (19.43); S, 7.37 (7.41).

### EXAMPLE 7

### Preparation of Adipate Salt of Compound 2

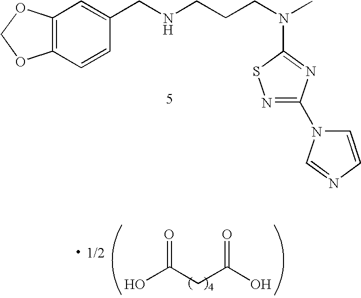

### Preparation of compound 5: N′-Benzo[1,3]dioxol-5-ylmethyl-N-(3-imidazol-1-yl-[1,2,4]thiadiazol-5-yl)-N-methyl-propane-1,3-diamine adipate salt

A suspension of 4 (15.3 g, 41.08 mmol) in EtOH (82 mL) was heated to 55° C. for 15 minutes to afford a clear solution. Adipic acid (3.06 g, 20.95 mmol) was added causing immediate precipitation. The suspension was stirred for an additional 15 minutes at 55° C. then n-heptane (164 mL) was added and the mixture was cooled to rt. The solid was collected by vacuum filtration and washed with n-heptanes (200 mL) to afford 15.62 g (85%) of 5 as a white solid. [M+H]⁺ 373.23; ¹H-NMR (400 MHz, CD₃OD) δ 8.36 (t, 1H), 7.75 (t, 1H), 7.08 (t, 1H), 6.88 (d, 1H), 6.83 (dd, 1H), 6.75 (d, 1H), 5.94 (s, 2H), 3.93 (s, 2H), 3.80-3.64 (br s, 2H), 3.14 (br s, 3H), 2.90 (m, 2H), 2.22 (m, 2H), 2.05 (quint, 2H), 1.62 (m, 2H);

Elemental found (calc) C, 53.73 (53.92); H, 5.61 (5.66); N, 18.62 (18.86); S, 7.11 (7.20).

### EXAMPLE 8

### Microscale Experiments to Produce Salts of Compound 1

Microscale experiments were carried out individually, and generally involved preparation of a solution containing equimolar amounts of benzo[1,3]dioxol-5-ylmethyl-{2-[2-(2-imidazol-1-yl-6-methyl-pyrimidin-4-yl)-pyrrolidin-1-yl]-ethyl}-amine (Compound 2, from a 125 mg/mL stock solution in methanol, or an oily residue thereof) and acid in a suitable solvent (methanol, acetonitrile, tetrahydrofuran, ethyl acetate, methyl tert-butyl ether (MTBE), toluene, and mixtures thereof), followed by addition of a suitable second solvent or antisolvent to facilitate precipitation, and/or evaporation (slow, fast, or flash), optionally accompanied by sonication. In the slow and fast evaporation modes, the sample vial was covered with aluminum foil pierced with one small or large (respectively) hole and allowed to evaporate slowly at ambient temperature; in the flash evaporation mode, the vial was covered with aluminum foil pierced with one large hole and allowed to evaporate quickly at ambient temperature, then rotovapped. Solids were recovered after various lengths of time, from immediately to three days after precipitation and/or evaporation, and characterized by techniques known in the art. It is expected that a screen performed with N′-(3-imidazol-1-yl-[1,2,4]thiadiazol-5-yl)-N′-thiazol-2-ylmethyl-propane-1,3-diamine would yield similar results.

Hydrochloride

Following combination of Compound 1 and hydrochloric acid in equimolar amounts in ethyl acetate, solids precipitated and solvent was removed by fast evaporation and analyzed.

Following combination of Compound 1 and hydrochloric acid in equimolar amounts in methanol and ethyl acetate, flash evaporation at 30° C. produced an oil, which was redissolved in ethyl acetate. Fast evaporation at room temperature produced an oil which was dissolved in methanol and ethyl acetate and fast-cooled from ˜70° C. to room temperature, yielding solids which were left stirring overnight at room temperature before being recovered and analyzed.

Following combination of Compound 1 and hydrochloric acid in equimolar amounts in methanol, both slow evaporation at room temperature and fast evaporation at 30° C. yielded a dark oil.

Following combination of Compound 1 and hydrochloric acid in equimolar amounts in 1:4 ethanol:ethyl acetate, the solution at 48.4 mg/mL was seeded with product crystals from the methanol/ethyl acetate experiment and stirred overnight. Solids were recovered by filtration, dried in vacuum oven, and analyzed.

Hydrobromide

Following combination of Compound 1 and hydrobromic acid in equimolar amounts in methanol and ethyl acetate, flash evaporation at 30° C. produced solids and oil. Precipitation was induced by addition of ethyl acetate with sonication, and solids were immediately recovered and analyzed.

Following combination of Compound 1 and hydrobromic acid in equimolar amounts in methanol, slow evaporation at room temperature produced oil and solids. Precipitation was induced by addition of ethyl acetate with sonication, and solids were recovered after one day in solvent and analyzed.

Following combination of Compound 1 and hydrobromic acid in equimolar amounts in methanol and methyl tert-butyl ether, flash evaporation at room temperature produced oil and solids. Precipitation was induced by addition of EtOAc with sonication, and solids were recovered after three days in solvent.

Oxalate

Following combination of Compound 1 and oxalic acid in equimolar amounts in isopropanol, flash evaporation at ˜30° C. produced an oil, which was redissolved in the same solvent. Fast evaporation at room temperature still yielded an oil. Similarly, combination of Compound 1 and oxalic acid in equimolar amounts in methanol followed by slow evaporation at room temperature yielded an oil.

Following combination of Compound 1 and oxalic acid in equimolar amounts in 10:1 methyl tert-butyl ether:methanol, precipitation was effected by solvent-antisolvent addition at ˜60° C. Slow evaporation yielded oil and solids (too few for analysis).

Acetate

Following combination of Compound 1 and acetic acid in equimolar amounts in isopropanol, flash evaporation at ˜30° C. produced an oil, which was redissolved in the same solvent. Fast evaporation at room temperature still yielded an oil. Similarly, combination of Compound 1 and acetic acid in equimolar amounts in methanol followed by slow evaporation at room temperature yielded an oil. Similarly, combination of Compound 1 and acetic acid in equimolar amounts in 15:1 methyl tert-butyl ether:methanol yielded an oil.

Phosphate

Following combination of Compound 1 and phosphoric acid in equimolar amounts in methanol and isopropanol, solvent-antisolvent addition yielded a solid which was lost during filtration.

Following combination of Compound 1 and phosphoric acid in equimolar amounts in methanol, slow evaporation at room temperature yielded an oil.

Following combination of Compound 1 and phosphoric acid in equimolar amounts in 10:3 toluene:methanol, slow cooling from ˜80° C. to room temperature resulted in an oil. Subsequent flash evaporation at ˜40° C. also yielded an oil.

Following combination of Compound 1 and phosphoric acid in equimolar amounts in 15:4 methylene chloride:methanol, precipitation occurred at ˜50° C. Slow evaporation at room temperature resulted in an oil.

Hippurate

Following combination of Compound 1 and hippuric acid in equimolar amounts in acetonitrile, flash evaporation at ˜30° C. produced an oil, which was redissolved in the same solvent. Fast evaporation at room temperature still yielded an oil. Similarly, combination of Compound 1 and hippuric acid in equimolar amounts in methanol followed by slow evaporation at room temperature yielded an oil.

Combination of Compound 1 and hippuric acid in equimolar amounts in 15:1 methyl tert-butyl ether:methanol followed by solvent-antisolvent addition resulted in a cloudy solution. Fast evaporation at room temperature yielded an oil.

Following combination of Compound 1 and hippuric acid in equimolar amounts in 10:1 nitromethane:methanol, slow cooling from ˜90° C. to room temperature with the vial open produced an orange solution. Fast evaporation at room temperature was still in progress.

### EXAMPLE 9

### Microplate Experiment to Produce Salts of Compound 2

Experiments were carried out in a 96-well, polypropylene-bottomed microplate. 50 μL aliquots of an approximately 40 mg/mL stock solution of N-benzo[1,3]dioxol-5-ylmethyl-N-(3-imidazol-1-yl-[1,2,4]thiadiazol-5-yl)-N-methyl-propane-1,3-diamine (Compound 1) in methanol were added to the wells of the microplate, which was centrivapped for about 2 minutes to remove the excess methanol leaving approximately 2 mg of compound free base. 15 μL of methanol was added to each well, followed by 55.9 μL of a 0.1 M solution of a given carboxylic acid in methanol, and the plate was allowed to evaporate overnight. 50 μL portions of either methanol, 95:5/ethanol:H₂0, isopropranol, and methylene chloride were then added. The microplate was sealed and maintained at approximately 55° C. for approximately 3 hours and cooled to ambient temperature. The solvent was subsequently allowed to evaporate in a fume hood. The samples were then recovered and examined using standard techniques known in the art.

Additional quantites of the hydrochloride, acetate and adipate salts of Compound 2 were prepared for characterization by techniques known in the art, including XRPD.

Hydrochloride

Compound 2 free base (157.49 mg) was reacted with a 0.1 M HCl (4360 L) solution at 55° C. in absolute, ethanol. An equal volume of antisolvent (heptane) was added to the reactant solution at 55° C. with stirring. The solution was allowed to reach room temperature and the solids were then filtered and recovered (36% yield).

A second attempt was made by taking Compound 2 free base (136.46 mg) and was warmed to 55° C. in absolute, ethanol. A slight excess of a 1.OM HCl solution in diethyl ether was added (405 L) after the solution was allowed to reach room temperature. An equal volume of antisolvent (diethyl ether) was added to the reactant solution at room temperature with stirring. The solids were filtered and recovered (68% yield).

A third attempt was made by taking Compound 2 free base (246.45 mg) and warming to 55° C. and dissolving in isopropanol. A slight excess of a 1.OM HCl solution in diethyl ether (730 L) was added after the solution was allowed to reach room temperature. Two volumes of antisolvent (hexanes) were added to the reactant solution at room temperature with stirring. The solids were filtered and recovered (87.1% yield).

Acetate

The Compound 2 free base (121.15 mg) was reacted with a slight excess of a 0.1 M acetic acid (3354 L) solution at 55° C. This solution was allowed to reach room temperature and allowed to slowly evaporate over night. The remaining solution was evaporated rapidly. The solid material was dissolved in ethanol and an equal volume of antisolvent (heptane) was added to the solution at 55° C. with stirring. The solution was allowed to reach room temperature and the solids were then filtered and recovered (18% yield).

A second attempt was made by taking the Compound 2 free base and warming to 55° C. and dissolving in ethanol. A slight excess of 0.1 M acetic acid was added. Two volumes of antisolvent (heptane) were added to the solution at 55° C. with stirring. No solids precipitated out of solution.

A third attempt was made by taking the Compound 2 free base (161.01 mg) and warming to 55° C. and dissolving in isopropanol. A slight excess of a 1.0 M acetic acid solution in isopropanol was added. Two volumes of antisolvent (hexanes) were added to the solution at 55° C. with stirring. The solution was allowed to reach room temperature and the solids were then filtered and recovered (84.9% yield).

Adipate

Compound 2 free base (174.13 mg) was reacted with a slight excess of a 0.1 M adipic acid 4821 L) solution at 55° C. with stirring in absolute ethanol. The solution was allowed to cool to room temperature and slowly evaporated for a full day. The remaining solution was rotovapped to dryness. The solid material was dissolved in ethanol at 55° C. and an equal volume of antisolvent was added. The solution was allowed to reach room temperature. The solids were recovered and filtered (49.7% yield).

Compound 2 free base was reacted with half an equivalent of a 0.1 M adipic acid (1700 iL) solution at 55° C. with stirring in absolute ethanol. Two volumes of antisolvent (heptane) were added to the solution at 55° C. The solution was then allowed to slowly cool to room temperature. The solids that precipitated out of solution were recovered and filtered (99.9% yield).

### EXAMPLE 10

### X-Ray Powder Diffraction Analysis of Compounds 1 and 2

2X-ray powder diffraction (XRPD) analysis of the microplate was performed using a Bruker D-8 Discover diffractometer and Bruker's General Area Diffraction Detection System (GADDS, v. 4.1.14). An incident beam of CuKα radiation was produced using a fine focus tube (40 kV, 40 mA), a Göbel mirror, and a 0.5 mm double-pinhole collimator. Samples were positioned for analysis by securing them to a translation stage and moving the sample to intersect the incident beam. Samples were analyzed in transmission mode using an incident—beam angle (θ₁) of 7° and a constant detector angle (2θ) of 20°. The incident beam was scanned ±6° relative to the well-bottom normal and rastered over a 0.4 mm×0.4 mm area of the sample during the analysis. Scanning and rastering the incident beam optimizes orientation statistics and maximizes the diffraction signal. A beam-stop was used to minimize air scatter and interference from the incident beam at low angles. Diffraction patterns were collected in 50 seconds using a Hi-Star area detector located 14.94 cm from the sample and processed using GADDS. The intensity in the GADDS image of the diffraction pattern was integrated from 2° to 37° 2θ and from 167° to −13°chi using a step size of 0.04°20. The integrated patterns display diffraction intensity as a function of 2θ. Prior to the analysis a NIST silicon SRM 640 c standard was analyzed to verify the Si 111 peak position is within ±0.05° 2θ of the NIST-certified value, 26.441° 2θ. The incident-beam intensity was verified to be >30% of the intensity generated by the newly installed tube. These analyses were performed under non-cGMP conditions.

X-ray powder diffraction (XRPD) analyses of scaled-up salts were performed using a Shimadzu XRD-6000 X-ray powder diffractometer using CuKα radiation. The instrument is equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A θ-2θ continuous scan at 3°/min (0.4 sec/0.02° step) from 2.5 to 40° 2θ was used. A silicon standard was analyzed to check the instrument alignment. Data were collected and analyzed using XRD-6000 v. 4.1. Samples were prepared for analysis by placing them in a sample holder.

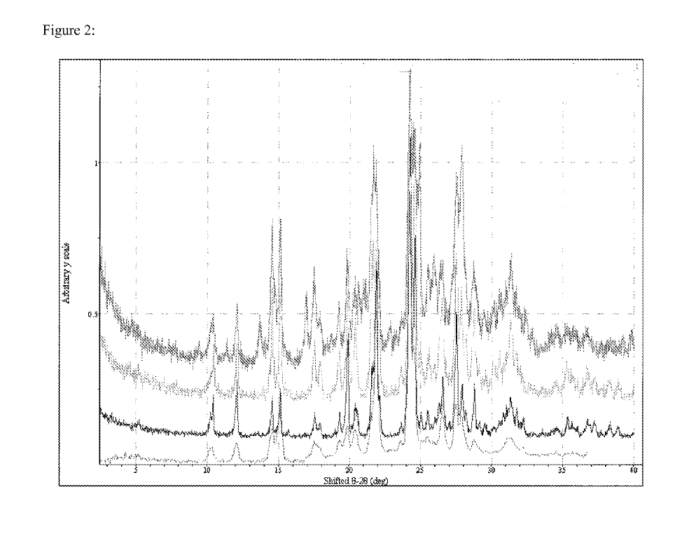

FIG. 2 compares the XRPD patterns obtained on material from the hydrochloride salt scale-up attempt to material obtained using the same solvent system in the microplate. Although other patterns were also observed in the plate, the similarity of these patterns indicates that the same solid form was prepared.

### EXAMPLE 11

### Structural and Stereochemical Resolution of Compound 2 Using X-Ray Crystallography

The hydrochloric acid salt of Compound 2 was used for this experiment instead of Compound 2 due to the unsuitability of the Compound 2 crystals for X-ray structure determination. The sample submitted for analysis contained numerous large, well formed rectangular blocks. One such block was trimmed to the dimensions 0.4×0.4×0.3 mm3, coated with mineral oil, picked up on a nylon loop and chilled to 100 K on the goniometer stage of a Bruker three-axis platform diffractometer equipped with an APEX detector and a Krvoflex low-temperature device. All software used in the subsequent data collection, processing and refinement is contained in libraries maintained by Bruker-AXS. Madison, Wis.

From sixty randomly chosen exposures taken in three sequences of twenty exposures at 0.3 deg intervals, it was possible to uniquely assign the crystal to the triclinic crystal system with the reported unit cell dimensions. The centrosymmetric space group P-i was initially chosen based on the statistical distribution of E-values and verified by the results of further dfata processing. The volume of the unit cell indicated that it contained two molecules.

A full hemisphere of data were collected at 100 K yielding 6.357 reflections of which 3.795 were crystallographically independent under triclinic symmetry providing up to a two-fold redundancy in coverage and a very low merging R factor. The data were first processed by SAINT, a program that integrated the 1,800 individual exposures and prepares a list of reflections and intensities. Corrections were made for absorption, polarization and Lorenzian distortion using SADABS. The structure was solved using direct methods (TREF) and subsequent difference maps were used to locate all non-hydrogen atoms. Refinement using SHELXTL routines for a model incorporating anisotropic thermal parameters for all non-hydrogen atoms and hydrogen atoms as idealized isotropic contributions resulted in a final structure with very low residuals and esd's for bond parameters. Table 1 presents the crystal data and structure refinement for Compound 2 hydrochloride salt. Table 2 presents the atomic coordinates (×10⁴) and equivalent isotropic displacement parameters (Å²×10³) for Compound 20 hydrochloric acid salt. U(eq) is defined as one third of the trace of the orthogonalized Ui tensor. Table 3 presents the bond angles for Compound 2.

TABLE 1

| Identification code | Compound 2 Hydrochloric acid salt |
|---|---|
| Empirical formula | C₁₇H₂₁ClN₆0₂S |
| Formula weight | 408.91 |
| Temperature | 100(2) K |
| Wavelength | 0.71073 Å |
| Crystal System | Triclinic |
| Space Group | p-1 |
| Unit Cell Dimensions | a = 6 35 16(14); Å α = 96.898(3)° |
| | b = 8 4820(18); Å β = 92.819(3)° |
| | c = 16987(4); Å γ = 91.726(3)° |
| Volume | 906.8(3) Å³ |
| Z | 2 |
| Density (calculated) | 1.498 g/cm³ |
| Absorption Coefficient | 0.353 mm⁻¹ |
| F(000) | 428 |
| Crystal Size | 0.40 × 0.40 × 0.30 mm³ |
| Theta range for data | 2.42 to 28.13° |
| collection | |
| Index Ranges | −8 ≦ h ≦ 8, −10 ≦ k ≦ 11, −22 ≦ l ≦ 18 |
| Reflections Collected | 6357 |
| Independent Reflections | 3795 [R(int) = 0.0197] |
| Completeness to | 85.40% |
| theta = 28.13° | |
| Absorption correction | None |
| Max. and min. transmission | 0.9015 and 0.8716 |
| Refinement method | Full-matrix least-squares on F² |
| Data/restraints/parameters | 3795/0/252 |
| Goodness-of-fit on F2 | 1.053 |
| Final R indices | R1 = 0 0382, wR2 = 0.1058 |
| [1 > 2sigma(1)]° | |
| R indices (all data) | R1 = 0 0398, wR2 = 0.1072 |
| Largest duff, peak and hole | 0.585 and −0.606 e Å³ |

TABLE 2

| | x | y | z | U(eq) |
|---|---|---|---|---|
| CI(1) | 1041(1) | 2307(1) | −330(1) | 14(1) |
| S(1) | 12382(1) | 921(1) | −872(1) | 18(1) |
| 0(1) | 4010(2) | 8243(1) | −5240(1) | 17(1) |
| 0(2) | 1647(2) | 7877(1) | −4284(1) | 19(1) |
| N(1) | 5925(2) | 2884(2) | −3048(1) | 15(1) |
| N(2) | 9018(2) | −559(2) | −1724(1) | 15(1) |
| N(3) | 8507(2) | 1398(2) | −642(1) | 15(1) |
| N(4) | 11875(2) | 2242(2) | −105(1) | 18(1) |
| N(5) | 8923(2) | 3343(2) | 481(1) | 16(1) |
| N(6) | 6525(2) | 4540(2) | 1220(1) | 22(1) |
| C(1) | 2192(3) | 8900(2) | −4861(1) | 18(1) |
| C(2) | 3914(2) | 6233(2) | −3520(1) | 16(1) |
| C(3) | 3478(2) | 7126(2) | −4122(1) | 15(1) |
| C(4) | 4904(2) | 7342(2) | −4695(1) | 14(1) |
| C(5) | 6850(2) | 6682(2) | −4694(1) | 16(1) |
| C(6) | 7337(2) | 5789(2) | −4072(1) | 16(1) |
| C(7) | 5911(2) | 5563(2) | −3495(1) | 15(1) |
| C(8) | 6497(2) | 4613(2) | −2833(1) | 16(1) |
| C(9) | 6425(3) | 1959(2) | −2375(1) | 16(1) |
| C(10) | 6119(3) | 186(2) | −2630(1) | 17(1) |
| C(11) | 6766(2) | −762(2) | −1952(1) | 16(1) |
| C(12) | 10513(3) | −1307(2) | −2262(1) | 19(1) |
| C(13) | 9710(2) | 513(2) | −1122(1) | 14(1) |
| C(14) | 9822(3) | 2300(2) | −103(1) | 15(1) |
| C(15) | 6828(3) | 3485(2) | 615(1) | 20(1) |
| C(16) | 8531(3) | 5124(2) | 1493(1) | 20(1) |
| C(17) | 10024(3) | 4402(2) | 1047(1) | 18(1) |

TABLE 3

| Bond | Bond Length and Angle |
|---|---|
| S(1)-N(4) | 1.6651(15) |
| S(1)-C(13) | 1.7428(16) |
| O(1)-C(4) | 1.3813(19) |
| O(1)-C(1) | 1.4436(19) |
| O(2)-C(3) | 1.3746(19) |
| O(2)-C(1) | 1.433(2) |
| N(1)-C(9) | 1.490(2) |
| N(1)-C(8) | 1.498(2) |
| N(2)-C(13) | 1.332(2) |
| N(2)-C(12) | 1.457(2) |
| N(2)-C(11) | 1.463(2) |
| N(3)-C(13) | 1.327(2) |
| N(3)-C(14) | 1.356(2) |
| N(4)-C(14) | 1.307(2) |
| N(5)-C(15) | 1.367(2) |
| N(5)-C(17) | 1.383(2) |
| N(5)-C(14) | 1.403(2) |
| N(6)-C(15) | 1.305(2) |
| N(6)-C(16) | 1.390(2) |
| C(2)-C(3) | 1.366(2) |
| C(2)-C(7) | 1.407(2) |
| C(3)-C(4) | 1.387(2) |
| C(4)-C(5) | 1.372(2) |
| C(5)-C(6) | 1.400(2) |
| C(6)-C(7) | 1.3.92(2) |
| C(7)-C(8) | 1.500(2) |
| C(9)-C(10) | 1.518(2) |
| C(10)-C(11) | 1.529(2) |
| C(16)-C(17) | 1.357(2) |
| N4)-S(1)-C(13) | 92.50(7) |
| C(4)-O(1)-C(1) | 104.26(12) |
| C(3)-O(2)-C(1) | 104.59(12) |
| C(9)-N(1)-C(8) | 111.43(13) |
| C(13)-N(2)-C(12) | 119.71(14) |
| C(13)-N(2)-C(11) | 120.78(13) |
| C(12)-N(2)-C(11) | 118.33(14) |
| C(13)-N(3)-C(14) | 106.94(14) |
| C(14)-N(4)-S(1) | 105.95(12) |
| C(15)-N(5)-C(17) | 107.03(14) |
| C(15)-N(5)-C(14) | 127.29(15) |
| C(17)-N(5)-C(14) | 125.67(14) |
| C(15)-N(6)-C(16) | 105.11(15) |
| O(2)-C(1)-O(1) | 106.59(12) |
| C(3)-C(2)-C(7) | 116.83(14) |
| C(2)-C(3)-O(2) | 128.09(14) |
| C(2)-C(3)-C(4) | 122.16(14) |
| O(2)-C(3)-C(4) | 109.71(14) |
| C(5)-C(4)-O(1) | 128.33(15) |
| C(5)-C(4)-C(3) | 122.35(15) |
| O(1)-C(4)-C(3) | 109.29(14) |
| C(4)-C(5)-C(6) | 116.19(15) |
| C(7)-C(6)-C(5) | 121.79(15) |
| C(6)-C(7)-C(2) | 120.66(15) |
| C(6)-C(7)-C(8) | 120.28(14) |
| C(2)-C(7)-C(8) | 119.06(14) |
| N(1)-C(8)-C(7) | 111.76(13) |
| N(1)-C(9)-C(10) | 111.12(13) |
| C(9)-C(10)-C(11) | 110.95(14) |
| N(2)-C(11)-C(10) | 112.43(13) |
| N3)-C13)-N2) | 125.66(15) |
| N(3)-C(13)-S(1) | 111.47(12) |
| N(2)-C(13)-S(1) | 122.87(12) |
| N(4)-C(14)-N(3) | 123.10(15) |
| N(4)-C(14)-N(5) | 118.81(15) |
| N(3)-C(14)-N(5) | 118.09(14) |
| N(6)-C(15)-N(5) | 111.85(15) |
| C(17)-C(16)-N(6) | 110.75(15) |
| C(16)-C(17)-N(5) | 105.26(15) |

### EXAMPLE 12

### Moisture Sorption/Desorption Analysis for Hygroscopicity of Compound 2

Moisture sorption/desorption data (FIG. 3) shows an initial weight loss for Compound 2 of approximately 0.16% upon equilibration at 5% RH. This weight was gradually regained by approximately 75% RH with a total weight gain of approximately 0.64% at 95% RH. Slightly more weight was lost during desorption with litle hysterisis. This behavior indicates the material is not hygroscopic.

### Bilogical Activity Assay

Enzyme Source

The source of nitric oxide synthase (NOS) enzyme can be generated in several ways including induction of endogenous iNOS using cytokines and/or lipopolysaccharide (LPS) in various cell types known in the art. Alternatively, the gene encoding the enzyme can be cloned and the enzyme can be generated in cells via heterologous expression from a transient or stable expression plasmid with suitable features for protein expression as are known in the art. Enzymatic activity (nitric oxide production) is calcium independent for iNOS, while the constitutive NOS isoforms, nNOS and eNOS, become active with the addition of various cofactors added to cellular media or extract as are well known in the art. Enzymes specified in Table 1 were expressed in HEK293 cells transiently transfected with the indicated NOS isoform.

DAN Assay

A major metabolic pathway for nitric oxide is to nitrate and nitrite, which are stable metabolites within tissue culture, tissue, plasma, and urine (S Moncada, A Higgs, N Eng J Med 329, 2002 (1993)). Tracer studies in humans have demonstrated that perhaps 50% of the total body nitrate/nitrite originates from the substrate for NO synthesis, L-arginine (P M Rhodes, A M Leone, P L Francis, A D Struthers, S Moncada, Biomed Biophys Res. Commun. 209, 590 (1995); L. Castillo et al., Proc Natl Acad Sci USA 90, 193 (1993). Although nitrate and nitrite are not measures of biologically active NO, plasma and urine samples obtained from subjects after a suitable period of fasting, and optionally after administration of a controlled diet (low nitrate/low arginine), allow the use of nitrate and nitrite as an index of NO activity (C Baylis, P Vallance, Curr Opin Nephrol Hypertens 7, 59 (1998)).

The level of nitrate or nitrite in the specimen can be quantified by any method known in the art which provides adequate sensitivity and reproducibility. A variety of protocols have also been described for detecting and quantifying nitrite and nitrate levels in biological fluids by ion chromatography (e.g., S A Everett et al., J. Chromatogr. 706, 437 (1995); J M Monaghan et al., J. Chromatogr. 770, 143 (1997)), high-performance liquid chromatography (e.g., M Kelm et al., Cardiovasc. Res. 41, 765 (1999)), and capillary electrophoresis (M A Friedberg et al., J. Chromatogr. 781, 491 (1997)). For example, 2,3-diaminonaphthalene reacts with the nitrosonium cation that forms spontaneously from NO to form the fluorescent product 1H-naphthotriazole. Using 2,3-diaminonaphthalene (“DAN”), researchers have developed a rapid, quantitative fluorometric assay that can detect from 10 nM to 10 μM nitrite and is compatible with a multi-well microplate format. DAN is a highly selective photometric and fluorometric reagent for Se and nitrite ion. DAN reacts with nitrite ion and gives fluorescent naphthotriazole (M C Carré et al., Analusis 27, 835-838 (1999)). Table 1 provides the test results of various compounds of the subject invention using the DAN assay.

A specimen can be processed prior to determination of nitrate or nitrite as required by the quantification method, or in order to improve the results, or for the convenience of the investigator. For example, processing can involve centrifuging, filtering, or homogenizing the sample. If the sample is whole blood, the blood can be centrifuged to remove cells and the nitrate or nitrite assay performed on the plasma or serum fraction. If the sample is tissue, the tissue can be dispersed or homogenized by any method known in the art prior to determination of nitrate or nitrite. It may be preferable to remove cells and other debris by centrifugation or another method and to determine the nitrate or nitrite level using only the fluid portion of the sample, or the extracellular fluid fraction of the sample. The sample can also be preserved for later determination, for example by freezing of urine or plasma samples. When appropriate, additives may be introduced into the specimen to preserve or improve its characteristics for use in the nitrate or nitrite assay.

The “level” of nitrate, nitrite, or other NO-related product usually refers to the concentration (in moles per liter, micromoles per liter, or other suitable units) of nitrate or nitrite in the specimen, or in the fluid portion of the specimen. However, other units of measure can also be used to express the level of nitrate or nitrite. For example, an absolute amount (in micrograms, milligrams, nanomoles, moles, or other suitable units) can be used, particularly if the amount refers back to a constant amount (e.g., grams, kilograms, milliliters, liters, or other suitable units) of the specimens under consideration. A number of commercially available kits can be used. Results are shown in Table 4 below.

TABLE 4

| Compound # | EC₅₀ hiNOS | EC₅₀ heNOS | EC₅₀ hnNOS |
|---|---|---|---|
| Compound 1 | <1 μM | >10 μM | >1 μM |
| Compound 2 | <1 μM | >10 μM | >1 μM |

This table is adapted from Table 1 in U.S. Application Publication No. US2005/0116515A1, which is herein incorporated by reference in its entirety.

### In Vivo Assays

Carrageenan Test

Injection of carrageenan subcutaneously into the hind foot (paw) of a rat induces robust inflammation and pain. The inflammatory response begins 1-2 hrs post-carrageenan injection and persists for at least five hours following inoculation. In addition, the rat's inflamed hind paw is sensitive to noxious (hyperaglesia) or innocuous (allodynia) stimuli, compared to the contralateral hind paw. Compounds can be evaluated in this model for anti-hyperalgesia and anti-inflammatory activity. A general increase in threshold or time to respond following drug administration suggests analgesic efficacy. A general decrease in paw swelling following drug administration suggests anti-inflammatory efficacy. It is possible that some compounds will affect the inflamed paw and not affect the responses of the contralateral paw.

Embodiments of the carrageenan foot edema test are performed with materials, reagents and procedures essentially as described by Winter, et al., (Proc. Soc. Exp. Biol. Med., 111, 544 (1962)). Male Sprague-Dawley rats were selected in each group so that the average body weight was as close as possible (175-200 g). The rats are evaluated for their responsiveness to noxious (paw pinch, plantar test) or innocuous (cold plate, von Frey filaments) stimuli.

In a prophylactic embodiment, following determination of “Pre-carrageenan” responses, a subplantar injection of the test compound or a placebo are administered. Following determination of “Pre-carrageenan” responses, the left hind paw of the rat is wrapped in a towel so that its right hind paw is sticking out. One hour thereafter, a subplantar injection of 100 μL of a 1% solution of carrageenan/sterile saline is injected subcutaneously into the plantar right hind paw, similar. Three hours (and optionally five hours) after carrageenan injection, the rats are evaluated for their responsiveness to noxious or innocuous stimuli and the paw volume was again measured. The paw withdrawal thresholds and average foot swelling in a group of drug-treated animals are compared with those of the group of placebo-treated animals and the percentage inhibition of pain and/or edema is determined (Otterness and Bliven, Laboratory Models for Testing NSAIDs, in Non-steroidal Anti-Inflammatory Drugs, (J. Lombardino, ed. 1985)).

In a therapeutic embodiment, following determination of “Pre-carrageenan” responses a subplantar injection of 100 μL of a 1% solution of carrageenan/sterile saline is administered. Two hours after carrageenan injection, the rats are evaluated for their responsiveness to noxious or innocuous stimuli and the paw volume is measured. Immediately following this testing, a subplantar injection of the test compound or a placebo was administered. Three hours and five hours after carrageenan injection (one and three hours after compound/placebo injection), the rats are evaluated for their responsiveness to noxious or innocuous stimuli and the paw volume is again measured. The paw withdrawal thresholds and average foot swelling in a group of drug-treated animals are compared with those of the group of placebo-treated animals and the percentage inhibition of pain and/or edema is determined.

Formalin Test

Subcutaneous injection of dilute formalin into the hind paw of a rat induces chronic pain. To test the efficacy of prophylactic and therapeutic agents, pain-related behaviors are observed over a period of time after introduction thereof. Biting, scratching, and flinching of the hind paw is measured to determine a response to the test compound. Typically, numerous biting and flinching behaviors are observed following formalin injection (“acute phase”), followed by a period of non-activity (10-15 minutes, “interphase”), followed by reemergence of pain behavior for the remainder of the test (15-60 minutes, “chronic phase”). Compared to saline-treated rats, rats treated with a typical analgesic such as morphine display fewer of these pain related behaviors.

Rats must weigh between 250-300 g and if naive should be handled once before running. Scrap rats may be used if they have had at least 5 days recovery, have no residual effects from previous procedures, and are within this weight range. Run subjects between 8:00-2:00 to minimize time of day effects in testing.

In a prophylactic embodiment, a subplantar injection of the test compound or a placebo was administered. One hour thereafter, a subcutaneous injection of 50 μL of a 5% formalin/sterile saline was administered. Pain related behaviors were then evaluated as described above.

In a therapeutic embodiment, a subcutaneous injection of 50 μL of a 5% formalin/sterile saline was administered. Fifteen minutes thereafter (i.e., during the “interphase”), a subplantar injection of the test compound or a placebo was administered. Pain related behaviors were then evaluated as described above.

Capsaicin Test

Subcutaneous injection of dilute capsaicin into the rat hind paw produces transient but pronounced hyperalgesia, allodynia and pain. This effect may be mitigated by pretreatment with a suitable agent, such as a topical anaesthetic or analgesic, and the extent of this mitigation quantified by evaluation of pain-related behaviors in response to noxious or innocuous stimuli as described above; rats pretreated with a known analgesic display fewer pain and allodynia related behaviors than controls. Compounds may be evaluated for their efficacy as potential analgesics in this manner as well.

Male Lewis rats weighing between 180 and 250 grams are used. The right hind paw is dipped into vehicle (100% acetone) or compound in vehicle for 30 seconds and then allowed to air-dry for 30 sec. To prevent the animal from licking the compound off the paw, the paw is wiped twice with a wet paper towel. At 15 min after application of vehicle or compound, 0.1 mg in 10 μL capsaicin is injected into right hind paw. Measurement of allodynia is performed 0.5 to 1 hour after capsaicin injection.

One procedure for quanitfying allodynia measures the rat behavioral response to presentation of von Frey filaments of increasing diameter. Each rat is placed in a small, clear cage on an elevated screen. Beginning with 4.31, the von Frey hair is presented perpendicularly to the right mid-plantar hind paw with sufficient force to cause slight buckling, for 6-8 seconds. If presentation lifts the hind paw it is disregarded, as it changes the nature of the stimulus. A positive response is noted if the paw is sharply withdrawn upon onset or offset of stimulus. Ambulation is considered an ambiguous response and the presentation is repeated. Stimuli are presented in a consecutive fashion. A positive response would call for the presentation of the immediately weaker weight filament next; likewise, no response would call for the immediately stronger. Presentations continue until a series of six consecutive responses from the first change is logged. The next rat is then tested. This procedure is standard in the art for the measurement of allodynia, but any other method known in the art which provides adequate sensitivity and reproducibility may be substituted.

Spinal Nerve Ligation Surgery

Neuropathy of dorsal spinal nerve roots L5 and L6 may be induced in rats. Kim S. H., and Chung J. M., An experimental model for peripheral neuropathy produced by segmental spinal nerve ligation in the rat. Pain 50: 355-363 (1992). Tight ligation of these nerve roots produces chronic neuropathic pain symptoms characterized by allodynia and hyperalgesia. The efficacy of potential analgesics on allodynia and hyperalgesia may be assessed in rats in a protocol and procedure described and adapted by T. Yaksh. Yaksh T. et al., Physiology and Pharmacology of Neuropathic Pain, Anesthesiology Clinics of North America, Vol. 14, Number 2 (1997) at pages 334 through 352.

Measuring Paw Volume (Edema)

Inflammation or edema may be quantified by measurement of paw volume (in ml), as injection of irritants such as CFA i.pl. results in an increase in paw volume as compared to an uninjected paw. Therefore, measurement of paw volume is a useful method for quantifying the ability of treatments to reduce inflammation in rats after administration of inflammatory agents.

This procedure is performed utilizing the UGO Basile Plethysmometer, which measures paw volume in ml. Setup involves filling the apparatus with solution, and then calibrating of the instrument. Solution should be changed every 2 to 3 days, and the calibration should be confirmed each time a test session is to be conducted. Detailed instructions regarding operation of the instrument are also included in the manual and will not be described here.

The procedure of paw volume measurement is simple. For each animal, the instrument should first be zeroed. Then the animal's irritated paw is placed into the measurement receptacle such that the entire paw up to the ankle is submerged. When the paw is submerged correctly and is restrained from movement, the foot pedal is pressed. This pedal serves as a signal to the instrument to measure change in volume in the measurement chamber (and therefore paw volume) at that moment. The animal is returned to its home cage, and the next animal is tested.

Occasionally, the measurement receptacle must be refilled to the top line, as repeated tests of animals gradually depletes the amount of solution in the instrument due to solution leaving the receptacle on animals' paws. The instrument may now be zeroed and is ready for more use.

Paw volume measurements generally are obtained before inflammatory introduction (baseline) and at several time points post-inflammation. Agents such as CFA, carrageenan, and capsaicin may be used, however, inflammation caused by these agents occur at different times.

LPS Challenge

Inhibition of induction of iNOS can be quantified via the LPS challenge. Inflammation, edema, and the onset of sepsis can be observed following an injection of lipopolysaccharide (LPS), a substance produced by Gram-negative bacteria. Injection of LPS has been shown to induce iNOS transcription, leading to measureable increases in both iNOS and NO. (Iuvone T et al., Evidence that inducible nitric oxide synthase is involved in LPS-mediated plasma leakage in rat skin through the activation of nuclear factor-κB, Br J Pharm 1998:123 1325-1330.) As described above, the level of nitric oxide in the specimen can be quantified by correlation with plasma nitrate or nitrite levels via chemiluminescence, fluorescence, spectophotometric assays, or by any method known in the art which provides adequate sensitivity and reproducibility, including those described above.

Male Lewis rats weighing 150-250 g are used in the studies. Rats may be fasted for up to 16 hours prior to the administration of LPS. Free access to water is maintained. Test compounds are administered with LPS or alone. Compounds are dissolved in the vehicle of 0.5% methycele/0.025% Tween 20 or 20% encapsin for oral administration. For the intravenous dosing, compounds are dissolved in saline or 0.5-3% DMSO/20% encapsin. The dosing volumes are 1-2 ml for oral and 0.3-1 ml for intravenous administration.

LPS is injected intravenously (under anesthesia) or intraperitoneally in sterile saline at a dose between 0.1-10 mg/kg in a volume not excess to 1 ml. The needle is 26-30 gauge. Following LPS injection, rats usually exhibit flu-like symptoms, principally involving lack of activity and diarrhea. In routine screening experiments, rats are sacrificed 1.5-6 hr after LPS injection and a terminal bleeding is performed under anesthesia to collect 1-3 ml blood samples and then animals are then euthanized by CO₂.

The following Table 5 lists compounds of the subject invention that were tested according to the above mentioned assays.

TABLE 5

| | | | Carrageenan | | |
|---|---|---|---|---|---|
| | | | Inflamed Pain | | Topical Capsaicin |
| | | | at 30 mg/kg, | LPS Induced | Allodynia |
| | | | (+), >40% | iNOS In | (+), >15% |
| | Formalin- | Chung- | inhibition | Vivo | inhibition |
| | Induced | Neuropathic | (−), <40% | (+), ED₅₀ <10 | (−), <15% |
| Compound | Pain | Pain | inhibition | (−), ED₅₀ >10 | inhibition |
| 1 | P < 0.01 | P < 0.001 | + | + | +at 0.5 hr |
| | at 25 mg/kg | at 25 mg/kg | | | +at 1 hr |
| 2 | P < 0.001 | P < 0.001 | − | + | +at 0.5 hr |
| | at 50 mg/kg | at 25 mg/kg | | | +at 1 hr |

This table is adapted from Table 2 in U.S. Application Publication No. US2005/0116515A1, which is herein incorporated by reference in its entirety.

### Solubility in Selected Solvents

The solubility of Compound 2 acetate was investigated with regard to potential process and formulation solvents. The solubility of Compound 2 acetate was evaluated by preparing saturated solution of Compound 2 in the selected solvents, filtering the samples (0.22 μm), diluting, and determining concentration by external standard HPLC using a rapid analysis assay. The solubility of Compound 2 acetate in various solvents and solvent mixtures is presented in Table 6.

TABLE 6

| Solvent | Solubility (mg/mL) |
|---|---|
| Water:Ethanol:Propylene Glycol (40:40:20) | 101 |
| Water | 44.6 |
| Propylene Glycol | 36.7 |
| Methanol | 58.3 |
| Ethanol | 8.5 |
| Isopropanol | 3.7 |
| Acetonitrile | 2.1 |
| Ethyl acetate | 2.0 |
| Dichloromethane | 10.0 |
| Hexanes | 0.003 |

The moisture sorption/desorption profiles of Compound 2 hydrochloride, Compound 2 acetate, and compound 2 adipate are presented in Table 7. Compounds were first equilibriated at 5% relative humidity, where some showed an initial weight loss. Relative humidity was then increased and weight measurements taken at regular intervals. Change is given as percent of original sample.

Compound 2 hydrochloride showed an initial weight loss of approximately <1% upon equilibration at 5% RH. This weight was gradually regained by approximately 75% RH with a total weight gain of approximately <5% at 95% RH. Slightly more weight was lost during desorption with little hysterisis. This behavior indicates the material is not hygroscopic.

Compound 2 acetate showed minimal weight gain over the range of 5 to 85% relative humidity (RH). Above 85% RH, KD7040 acetate gained substantial weight indicating that the compound is significantly hygroscopic at high RH. Most of the weight was lost on desorption with minor hysteresis.

Compound 2 adipate showed an initial weight loss of less than 1% upon equilibration at 5% RH. This weight was gradually regained by approximately 45% RH with a total weight gain of over 6% at 95% RH. This amount of weight was lost during desorption with no hysterisis. This behavior indicates the material is hygroscopic, especially at elevated humidities.

TABLE 7

| Percent | Weight % Change, | | Weight % Change, | | Weight % Change, | |
|---|---|---|---|---|---|---|
| Relative | Compound 2 HCl | | Compound 2 Acetate | | Compound 2 Adipate | |
| Humidity | Adsorption | Desorption | Adsorption | Desorption | Adsorption | Desorption |
| 5 | <−1 | <−2 | <1 | <1 | <0 | <0 |
| 15 | <−1 | <−1 | <1 | <1 | <0 | <0 |
| 25 | <−1 | <−1 | <1 | <1 | <0 | <0 |
| 35 | <−1 | <−1 | <1 | <1 | <0 | <0 |
| 45 | <−1 | <−1 | <1 | <1 | <1 | <1 |
| 55 | <−1 | <0 | <1 | <1 | <1 | <1 |
| 65 | <0 | <0 | <1 | <1 | <1 | <1 |
| 75 | <0 | <1 | <1 | <1 | <2 | <2 |
| 85 | <1 | <2 | <3 | <3 | <3 | <3 |
| 95 | <5 | <5 | <5 | <5 | >6 | >6 |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. An acetate salt of an iNOS inhibitor.

2. A salt of a compound of either Formula II 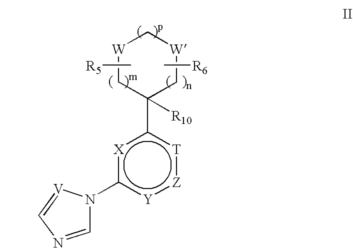
wherein:
T, V, X, and Y are independently selected from the group consisting of CR⁴ and N;
Z is from the group consisting of CR³ and N;
W and W′ are independently selected from the group consisting of CH₂, CR⁷R⁸, NR⁹, O, N(O), S(O)_{q} and C(O);
n, m and p are independently an integer from 0 to 5;
q is 0, 1, or 2;
R³, R⁴, R¹⁰, R¹⁴, R¹⁵, R¹⁶, R¹⁷ and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;
R⁵, R⁶, R⁷, R⁸, and R⁹ are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally optionally substituted heteroaralkyl, optionally substituted alkene, optionally substituted alkyne, —(O)N(R¹¹)R¹², —P(O)[N(R¹¹)R¹²]₂, —SO₂NHC(O)R¹¹, —N(R¹¹)SO₂R¹², —SO₂N(R¹¹)R¹², —NSO₂N(R¹¹)R¹², —C(O)NHSO₂R¹¹, —CH═NOR¹¹, —OR¹¹, —S(O)ₜ—R¹¹, —N(R¹¹)R¹², —N(R¹¹)C(O)N(R¹²)R¹³, —N(R¹¹)C(O)OR¹², —N(R¹¹)C(O)R¹², —[C(R¹⁴)R¹⁵]ᵣ—R¹², —[C(R¹⁴)R¹⁵]ᵣ—C(O)OR¹¹, —[C(R¹⁴)R¹⁵]ᵣ—[C(O)OR¹¹]₂, —[C(R¹⁴)R¹⁵]ᵣC(O)N(R¹¹)R¹², —[C(R¹⁴)R¹⁵]ᵣ—N(R¹¹)R¹², —[C(R¹⁴)R¹⁵]ᵣ—N(R¹¹)—[C(R¹⁴)R¹⁵]ᵣ R¹², —[C(R¹⁴)R¹⁵]ᵣ—OR¹¹, —N(R¹¹)—[C(R¹⁴)R¹⁵]ᵣ—R¹², —N(R¹¹)C(O)N(R¹³)—[C(R¹⁴)R¹⁵]ᵣ—R¹², —C(O)—[C(R¹⁴)R¹⁵]ᵣ—N(R¹¹)R¹², —N(R¹³)C(O) L (R¹¹)R¹², N(R¹¹) [C(R¹⁴)R¹⁵]ᵣ L R¹², N(R¹¹)C(O)N(R¹¹) [C(R¹⁴)R¹⁵]ᵣ L R¹², —[C(R¹⁴)R¹⁵]ᵣ-L-R¹², and -L-C(O)N(R¹¹)R¹²; or R⁵ and R⁶ together may form an optionally substituted aryl, optionally substituted heteroaryl, optionally substituted cycloalkyl, or optionally substituted heterocycloalkyl;
t is an integer from 0 to 2;
r is an integer from 0 to 5;
L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group;
R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)N(R¹⁷)R¹⁸, —[C(R¹⁴)R¹⁵]ᵣ—N(R¹⁷)C(O)OR¹⁸, —[C(R¹⁴)R¹⁵]ᵣ—R¹⁷, and —[C(R¹⁴)R¹⁵]ᵣ—N(R¹⁷)C(O)R¹⁸; or R¹¹ or R¹² may be defined by a structure selected from the group consisting of 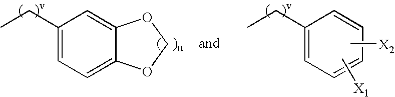
wherein:
u and v are independently an integer from 0 to 3; and
X¹ and X² are independently selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl;
or of Formula IV 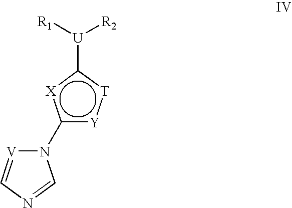
wherein:
T, X, and Y are independently selected from the group consisting of CR⁴, N, NR⁴, S, and O;
U is CR¹⁰ or N;
V is CR⁴ or N;
R¹ and R² are independently selected from the group consisting of hydrogen, halogen, optionally substituted alkyl, optionally substituted alkoxy, haloalkyl, haloalkoxy, optionally substituted aralkyl, optionally substituted aryl, optionally substituted heteroaryl, optionally optionally substituted heteroaralkyl, optionally substituted alkene, optionally substituted alkyne, —(O)N(R¹¹)R¹², —P(O)[N(R¹¹)R¹²]₂, —SO₂NHC(O)R¹¹, —N(R¹¹)SO₂R¹², —SO₂N(R¹¹)R¹², —NSO₂N(R¹¹)R¹², —C(O)NHSO₂R¹¹, —CH═NOR¹¹, —OR¹¹, S(O)ₜ—R¹¹, —N(R¹¹)R¹², —N(R¹¹)C(O)N(R¹²)R¹³, —N(R¹¹)C(O)OR¹², —N(R¹¹)C(O)R¹², —[C(R¹⁴)R¹⁵]ᵣ—R¹², —[C(R¹⁴)R¹⁵]ᵣ—C(O)OR¹¹, —[C(R¹⁴)R¹⁵]ᵣ—[C(O)OR¹¹]₂, —[C(R¹⁴)R¹⁵]ᵣC(O)N(R¹¹)R¹², —[C(R¹⁴)R¹⁵]ᵣ—N(R¹¹)R¹², —[C(R¹⁴ )R¹⁵]ᵣ—N(R¹¹)—[C(R¹⁴) R¹⁵]ᵣ R¹², —[C(R¹⁴)R¹⁵]ᵣ—N(R¹¹)—C(O)N(R¹¹)R¹², —[C(R¹⁴)R¹⁵]ᵣ—N(R¹¹)S(O)ₜ—C(O)N(R¹¹)R¹², —[C(R¹⁴)R¹⁵]ᵣ—OR¹¹, —N(R¹¹)—[C(R¹⁴)R¹⁵]ᵣ—R¹², —N(R¹¹)C(O)N(R¹³)—[C(R¹⁴)R¹⁵]ᵣR¹², —C(O)—[C(R¹⁴)R¹⁵]ᵣ—N(R¹¹)R¹², —N(R¹³)C(O)-L-(R¹¹)R¹², —N(R¹¹)—[C(R¹⁴)R¹⁵]ᵣ-L-R¹², —N(R¹¹)C(O)N(R¹¹)—[C(R¹⁴)R¹⁵]ᵣ-L-R¹², —[C(R¹⁴)R¹⁵]ᵣ-L-R¹², and -L-C(O) N(R¹¹)R¹²; or R⁵ and R⁶ together may form an optionally substituted aryl, optionally substituted heteroaryl, optionally substituted cycloalkyl, or optionally substituted heterocycloalkyl;
t is an integer from 0 to 2;
r is an integer from 0 to 5;
L is selected from the group consisting of an optionally substituted 3- to 7-membered carbocyclic group, an optionally substituted 3- to 7-membered heterocyclic group, an optionally substituted 6-membered aryl group, and an optionally substituted 6-membered heteroaryl group;
R⁴, R¹⁰, R¹⁴, R¹⁵, R¹⁶, R¹⁷, and R¹⁸ are independently selected from the group consisting of hydrogen, halogen, optionally substituted alkyl, optionally substituted haloalkyl, haloalkoxy, optionally substituted aralkyl, optionally substituted aryl, optionally substituted heteroaryl, optionally substituted heteroaralkyl, optionally substituted alkene, optionally substituted alkyne; or R¹⁴ and R¹⁵ may together form a carbonyl, optionally substituted carbocycle or optionally substituted heterocycle; or R¹⁴ and R¹⁵ together may be null, forming an additional bond;
R¹¹, R¹², and R¹³ are independently selected from the group consisting of hydrogen, halogen, optionally substituted alkyl, haloalkyl, haloalkoxy, optionally substituted aralkyl, optionally substituted aryl, optionally substituted heteroaryl, optionally substituted heteroaralkyl, optionally substituted alkene, optionally substituted alkyne, —OR¹⁷, —S(O)ₜR¹⁷, —[C(R¹⁴)R¹⁵]ᵣ—C(O)OR¹⁷, —[C(R¹⁴)R¹⁵]ᵣ—N(R¹⁷)R¹⁸, —[C(R¹⁴)R¹⁵]ᵣ—N(R¹⁶)C(O)(R¹⁷)R¹⁸, —[C(R¹⁴)R¹⁵]ᵣ—N(R¹⁷)C(O)OR¹⁸, —[C(R¹⁴)R¹⁵]ᵣ—R¹⁷, and —[C(R¹⁴)R¹⁵]ᵣ—N(R¹⁷)C(O)R¹⁸; or R¹¹ or R¹² may be defined by a structure selected from the group consisting of 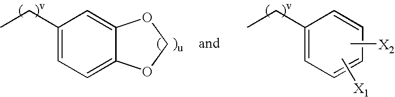
wherein:
u and v are independently an integer from 0 to 3; and
X¹ and X² are independently selected from the group consisting of hydrogen, halogen, hydroxy, lower acyloxy, optionally substituted lower alkyl, optionally substituted lower alkoxy, lower haloalkyl, lower haloalkoxy, and lower perhaloalkyl; or X¹ and X² together may form an optionally substituted aryl, optionally substituted heteroaryl, optionally substituted cycloalkyl, or optionally substituted heterocycloalkyl.

3. The salt as recited in claim 2, wherein said formula is Formula II.

4. The salt as recited in claim 3, wherein said compound is Compound 1.

5. The salt as recited in claim 2, wherein said formula is Formula IV.

6. The salt as recited in claim 2, wherein said salt is selected from the group consisting of hydrochloride, hydrobromide, acetate, trifluoroacetate, adipate, oxalate, phosphate, and hippurate.

7. The salt as recited in claim 6, wherein said salt is selected from the group consisting of hydrochloride, acetate, and adipate.

8. The salt as recited in claim 7, wherein said salt is acetate.

9. The salt as recited in claim 5, wherein said compound is Compound 1.

10. The salt as recited in claim 6, wherein said compound is Compound 2.

11. A salt of N′-benzo[1,3]dioxol-5-ylmethyl-N-(3-imidazol-1-yl-[1,2,4]thiadiazol-5-yl)-N-methyl-propane-1,3-diamine.

12. The salt as recited in claim 11, wherein said salt is selected from the group consisting of hydrochloride, acetate, and adipate.

13. N′-benzo[1,3]dioxol-5-ylmethyl-N-(3-imidazol-1-yl-[1,2,4]thiadiazol-5-yl)-N-methyl-propane-1,3-diamine acetate.

14. N′-benzo[1,3]dioxol-5-ylmethyl-N-(3-imidazol-1-yl-[1,2,4]thiadiazol-5-yl)-N-methyl-propane-1,3-diamine hydrochloride.

15. N′-benzo[1,3]dioxol-5-ylmethyl-N-(3-imidazol-1-yl-[1,2,4]thiadiazol-5-yl)-N-methyl-propane-1,3-diamine adipate.

16. The salt as recited in claim 3, formulated for topical administration.

17. The salt as recited in claim 2, for use as a medicament.

18. The salt as recited in claim 2, useful for the treatment or prevention of an iNOS-mediated disease.

19. A method for achieving an effect in a patient comprising the administration of a therapeutically effective amount of a salt as recited in claim 2 to a patient, wherein the effect is selected from the group consisting of inhibition if iNOS and treatment of an iNOS-mediated disease.

20. The method as recited in claim 19, wherein said disease is selected from the group consisting of inflammation, inflammatory pain, neuropathic pain, post-herpetic neuralgia, post-surgical pain, and an ocular disease.
